(19) United States
(12) Patent Application Publication
(10) Pub. No.: US 2013/0058879 A1
(43) Pub. Date: Mar. 7, 2013

(54) TRIAZINES AS REACTION ACCELERATORS

(71) Applicants: Rene Peter Scheurich, Gross-Zimmern (DE); Thomas Rudolph, Darmstadt (DE)

(72) Inventors: Rene Peter Scheurich, Gross-Zimmern (DE); Thomas Rudolph, Darmstadt (DE)

(73) Assignee: MERCK PATENT GESELLSCHAFT MIT BESCHRANKTER HAFTUNG, Darmstadt (DE)

(21) Appl. No.: 13/697,079

(22) PCT Filed: Apr. 13, 2011
(86) PCT No.: PCT/EP2011/001872

§ 371 Date: Nov. 9, 2012

(30) Foreign Application Priority Data
May 12, 2010 (EP) 10005033.5

(51) Int. Cl. A61K 8/49; G02B 5/22; A61Q 5/06; C09K 15/30; C07C 45/29; A61Q 17/04

(52) U.S. Cl. 424/59; 568/322; 424/70.1; 514/785; 514/788; 252/403; 252/589

(57) ABSTRACT

The invention relates to the use of at least one specific triazine or melamine and/or salt thereof as accelerator for a photoinduced reaction of 3-(4-tert-butylphenyl)-3-hydroxy-1-(4-methoxyphenyl)-propan-1-one, 1-(4-tert-butylphenyl)-3-hydroxy-3-(4-methoxyphenyl)-propan-1-one or a mixture of these compounds to give avobenzone. The invention furthermore relates to a composition comprising at least one specific triazine or melamine and to a method for the stabilisation of the UV absorption capacity of a composition by means of a mixture of at least one specific triazine or melamine together with 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or a mixture of these compounds, particularly preferably together with 3-(4-tert-butylphenyl)-3-hydroxy-1-(4-methoxyphenyl)propan-1-one.

The invention relates to the use of at least one compound of the formula I and/or salt thereof as accelerator for a photoinduced reaction of 3-(4-tert-butylphenyl)-3-hydroxy-1-(4-methoxyphenyl)propan-1-one, 3-(4-methoxyphenyl)-3-hydroxy-1-(4-tert-butylphenyl)propan-1-one (or synonymously 1-(4-tert-butylphenyl)-3-hydroxy-3-(4-methoxyphenyl)propan-1-one) or mixtures of these two compounds to give 1-(4-tert-butylphenyl)-3-(4-methoxyphenyl)propane-1,3-dione. The compound 1-(4-tert-butylphenyl)-3-(4-methoxyphenyl)propane-1,3-dione is also called avobenzone below. The invention furthermore relates to a composition comprising at least one compound of the formula I and to a method for the stabilisation of the UV absorption capacity of a composition by means of a mixture of at least one compound of the formula I together with 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or a mixture of these two compounds.

The following statements regarding one of the substances 3-(4-tert-butyl-phenyl)-3-hydroxy-1-(4-methoxyphenyl)propan-1-one or 1-(4-tert-butyl-phenyl)-3-hydroxy-3-(4-methoxyphenyl)propan-1-one also always apply correspondingly to the second substance which is not mentioned or to mixtures of the two substances.

Solar radiation or also radiation from artificial sources, in particular radiation having wavelengths of 280 to 400 nanometres, so-called UV radiation, can interact with ingredients of compositions and with materials. Ingredients or materials of this type can be compounds such as, for example, pigments, UV filters, antioxidants or plastics as well as polymers. Owing to the absorption of radiation, however, ingredients or materials of this type may experience a structural change through, for example, a photochemical reaction. A photochemical degradation of this type of ingredients or materials usually results in undesired impairment of the composition or material. In the case of dyes, for example, a photochemical degradation of this type may result in bleaching or a loss of gloss, and in the case of packaging materials, the plastic present may become brittle and thus lose its protective function.

Furthermore, the human skin undergoes certain aging processes, which are in some cases attributable to exogenous factors. The exogenous factors include, in particular, sunlight or artificial radiation sources having a comparable spectrum, as well as compounds which may arise due to the radiation, such as undefined reactive photoproducts, which may also have a free-radical or ionic character.

Furthermore, both the skin and also ingredients and materials are subjected to a certain oxidative stress due to the environment. This oxidative stress results in oxidative degradation, which may in turn likewise result in undesired impairment of the materials or compositions and which may reinforce the aging process of the skin.

A multiplicity of organic and inorganic UV filters and antioxidants are known which are able to absorb UV radiation and scavenge free radicals. These UV filters and antioxidants are consequently capable of protecting the human skin.

Owing to photochemical decomposition of the UV filters themselves by, for example, solar radiation or by artificial radiation sources having a comparable spectrum, however, the concentrations of the UV filters continuously drops during application or use, and consequently so does the protection against aggressive UV radiation. This effect can usually be countered by particularly stable UV filters, but these UV filters may also undergo a certain photochemical degradation. In addition, the stability of the respective UV filter is also dependent on the respective composition, meaning that adequate stability of the respective UV filter does not arise in every formulation.

The present invention is concerned with the problem of improving the protection, in particular in/or by compositions, against sunlight or sun-like light and/or against oxidative damage.

This problem is solved in accordance with the invention by the subject-matters of the independent claims. Advantageous embodiments are the subject-matter of the dependent claims.

Surprisingly, it has now been found that compounds of the formula I, thus mono- or polysubstituted triazines or melamines, can act as accelerators for a photoinduced reaction of 3-(4-tert-butylphenyl)-3-hydroxy-1-(4-methoxyphenyl)propan-1-one or 1-(4-tert-butylphenyl)-3-hydroxy-3-(4-methoxyphenyl)propan-1-one.

Photoinduced reaction here is taken to mean the conversion of, for example, 3-(4-tert-butylphenyl)-3-hydroxy-1-(4-methoxyphenyl)propan-1-one into avobenzone on irradiation with sunlight or sun-like light, where, in accordance with reaction scheme A below

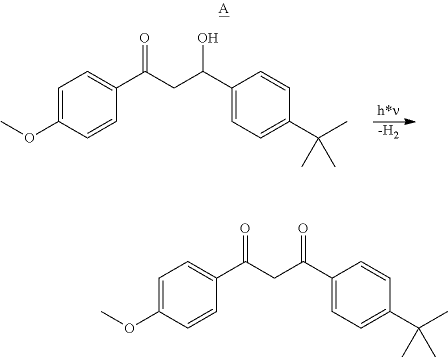

hydrogen is released, which is usually scavenged “in situ” by surrounding compounds.

Thus, the use of compounds of the formula I enables the formation of the UV filter avobenzone to be accelerated by photoinduced reaction of 3-(4-tert-butylphenyl)-3-hydroxy-1-(4-methoxyphenyl)propan-1-one or 1-(4-tert-butylphenyl)-3-hydroxy-3-(4-methoxyphenyl)propan-1-one or mixtures of these two compounds and therefore enables decreasing UV absorption capacity owing to decomposition of UV filters, for example in compositions, to be at least partially countered owing to the accelerated formation of avobenzone.

An accelerator is thus taken to mean a compound, in particular of the formula I, as described below, which accelerates a reaction of a starting material to give a product, so that the product is formed more quickly in the presence of an accelerator than in the absence thereof.

Furthermore, 3-(4-tert-butylphenyl)-3-hydroxy-1-(4-methoxyphenyl)propan-1-one can, in purely formal terms, release hydrogen owing to the photoinduced reaction to give avobenzone and thus exhibit a certain antioxidative action, meaning that 3-(4-tert-butylphenyl)-3-hydroxy-1-(4-methoxy-phenyl)propan-1-one together with a compound of the formula I can also be employed as antioxidant, in particular as photoantioxidant.

A photoantioxidant is thus taken to mean a compound which exhibits an antioxidant action on irradiation, in particular with light having a wavelength of 280 to 400 nanometres.

Hereinbelow, in each case independently of one another:

Alk¹ stands for a straight-chain or branched C₁- to C₂₀-alkyl group or for a straight-chain or branched C₂- to C₂₀-alkenyl group, which has at least one double bond, or for a straight-chain or branched C₂- to C₂₀-alkynyl group, which has at least one triple bond, and/or in which at least one or more non-adjacent C atoms of the alkyl, alkenyl or alkynyl group may be replaced by 0 or trimethylsilyl and/or which may contain at least one OH bonded to a primary or secondary C atom;
Alk² stands for a straight-chain or branched C₁- to C₁₂-alkyl group, which may be interrupted by at least one 0, Alk³ stands for a straight-chain or branched C₁- to C₁₂-alkyl group, in which at least one or more non-adjacent C atoms may be replaced by O and/or which may contain at least one OH bonded to a primary or secondary C atom,
Alk⁴ stands for a straight-chain or branched C₁- to C₈-alkyl group;
Cyc¹ stands for a C₃- to C₈-cycloalkyl group, which may have at least one double bond, and/or in which at least one CH₂ may be replaced by O or NH;
Cyc² stands for a C₅- to C₈-cycloalkyl group, which may have at least one double bond, and/or in which at least one CH₂ may be replaced by O or NH;
Arl¹ stands for an unsubstituted, mono- or polysubstituted C₆- to C₂₀-aryl group;
Arl² stands for an unsubstituted, mono- or polysubstituted C₆- to C₁₅-aryl group;
Arl³ stands for an unsubstituted, mono- or polysubstituted C₆- to C₁₀-aryl group;
Het¹ stands for an unsubstituted, mono- or polysubstituted C₅- to C₂₀-aryl group, in which at least one CH₂ has been replaced by O, S or NH and thus stands for an unsubstituted, mono- or polysubstituted C₅- to C₂₀-heteroaryl group,
Het² stands for an unsubstituted, mono- or polysubstituted C₅- to C₁₀-aryl group, in which at least one CH₂ has been replaced by O, S or NH and thus stands for an unsubstituted, mono- or polysubstituted C₅- to C₁₀-heteroaryl group;
Het³ stands for an unsubstituted, mono- or polysubstituted C₅- to C₆-aryl group, in which at least one CH₂ has been replaced by O, S or NH and thus stands for an unsubstituted, mono- or polysubstituted C₅- to C₆-heteroaryl group;
Eth¹ stands for —(CH₂—CH₂—O)ₘ₁-Alk², where 1≦m1≦16; where Alk² has a meaning described above,
Eth² stands for —(CH₂—CH₂—O)ₘ₁-Alk⁴, where 1≦m1≦8; where Alk⁴ has a meaning described above,
Eth³ stands for —(CH₂—CH₂—O)ₘ₁-Alk⁶, where 1≦m1≦4; where Alk⁶ has a meaning described above,
Hal stands for F, Cl, Br or I.

A straight-chain or branched C₁-C₄-alkyl group here includes methyl, ethyl, propyl-, isopropyl, butyl, x-methylpropyl (x=1; 2) and tert-butyl;

a straight-chain or branched C₁-C₈-alkyl group here includes the substituents of the C₁-C₄-alkyl group and pentyl, hexyl, heptyl, octyl, x-methylbutyl (x=1; 2; 3), x-methylpentyl (x=1; 2; 3; 4), x-methylhexyl (x=1; 2; 3; 4; 5), x-ethyl-pentyl (x=1; 2; 3), x-ethylhexyl (x=1; 2; 3; 4);
a straight-chain or branched C₁-C₁₂-alkyl group here includes the substituents of the C₁-C₈-alkyl group and nonyl, decyl, undecyl, dodecyl;
a straight-chain or branched C₁-C₂₀-alkyl group here includes the substituents of the C₁-C₁₂-alkyl group and tridecyl, tetradecyl, pentadecyl, hexadecyl, heptadecyl, octadecyl, nonadecyl, eicosyl;
a straight-chain or branched C₂-C₄-alkenyl group here includes ethenyl, x-propenyl (x=1; 2), x-butenyl (x=1; 2; 3) and butadienyl;
a straight-chain or branched C₂-C₈-alkenyl group here includes the substituents of the C₂-C₄-alkenyl group and x-pentenyl (x=1; 2; 3; 4), x-hexenyl (x=1; 2; 3; 4; 5), x-heptenyl (x=1; 2; 3; 4; 5; 6), x-octenyl (x=1; 2; 3; 4; 5; 6; 7);
a straight-chain or branched C₂-C₁₂-alkenyl group here includes the substituents of the C₂-C₈-alkenyl group and x-nonenyl (x=1; 2; 3; 4; 5; 6; 7; 8), x-decenyl (x=1; 2; 3; 4; 5; 6; 7; 8; 9), x-undecenyl (x=1; 2; 3; 4; 5; 6; 7; 8; 9; 10), x-dodecenyl (x=1; 2; 3; 4; 5; 6; 7; 8; 9; 10; 11);
a straight-chain or branched C₂-C₂₀-alkenyl group here includes the substituents of the C₂-C₁₂-alkenyl group and 9-octadecenyl, 9,12-octadecadienyl, 9,12,15-octadecatrienyl, 10-nonadecenyl, 10,13-nonadecadienyl, 10,13,16-nonadecatrienyl, 11-eicosenyl, 11,14-eicosadienyl, 11,14,17-eicosatrienyl;
a straight-chain or branched C₂-C₄-alkynyl group here includes acetylenyl, x-propynyl (x=1; 2), x-butynyl (x=1; 2; 3) and butadiynyl;
a straight-chain or branched C₂-C₈-alkynyl group here includes the substituents of the C₂-C₄-alkynyl group and x-pentynyl (x=1; 2; 3; 4), x-hexynyl (x=1; 2; 3; 4; 5), x-heptynyl (x=1; 2; 3; 4; 5; 6), x-octynyl (x=1; 2; 3; 4; 5; 6; 7);
a straight-chain or branched C₂-C₁₂-alkynyl group here includes the substituents of the C₂-C₈-alkynyl group and x-nonynyl (x=1; 2; 3; 4; 5; 6; 7; 8), x-decynyl (x=1; 2; 3; 4; 5; 6; 7; 8; 9), x-undecynyl (x=1; 2; 3; 4; 5; 6; 7; 8; 9; 10), x-dodecynyl (x=1; 2; 3; 4; 5; 6; 7; 8; 9; 10; 11);
a straight-chain or branched C₂-C₂₀-alkynyl group here includes the substituents of the C₂-C₁₂-alkynyl group and x-octadecynyl (x=1; 17), x-nonadecynyl (x=1; 18), x-eicosynyl (x=1; 19);
a C₅-C₆-cycloalkyl group here includes cyclopentyl, x-cyclopentenyl (x=1; 2; 3), x,y-cyclopentadienyl (x,y=1,3; 1,4; 2,4), cyclohexyl, x-cyclohexenyl (x=1; 2; 3), x,y-cyclohexadienyl (x,y=1,3; 1,4; 2,4; 2,5), x-tetrahydrofuranyl (x=2; 3), x-(2,5-dihydrofuranyl) (x=2; 3), x-tetrahydropyrrolyl (x=2; 3), x-(2,5-dihydropyrrolyl) (x=2; 3), x-(4,5-dihydrooxazolyl) (x=2; 4; 5), x-oxazolidinyl (x=2; 3; 4; 5), x-piperidinyl (x=2; 3; 4; 5), x-pyranyl (x=2; 3; 4; 5), x-(4,5-dihydropyranyl) (x=2; 3; 4; 5), x-piperazinyl (x=2; 3), x-(1,3-dioxanyl) (x=2; 4; 5; 6) and x-(1,4-dioxanyl) (x=2; 3; 5; 6);
a C₃-C₈-cycloalkyl group here includes the substituents of the C₅-C₆-cycloalkyl group and cyclopropyl, x-cyclopropenyl (x=1; 2), cyclobutanyl, x-cyclobutenyl (x=1; 2; 3), cycloheptyl, cyclooctyl, x-methylcylclohexyl (x=2; 3; 4), x-ethylcyclohexyl (x=2; 3; 4) and x,y-dimethylcyclohexyl (x,y=2,3; 2,4);
an unsubstituted, mono- or polysubstituted C₆-C₁₀-aryl group here includes, phenyl, naphthyl, x-methylphenyl (x=o,m,p), x-ethylphenyl (x=o,m,p), x-propylphenyl (x=o,m,p), x-isopropylphenyl (x=o,m,p), x-tert-butylphenyl (x=o,m,p), x-nitrophenyl (x=o,m,p), x-methoxyphenyl (x=o,m,p), x-ethoxyphenyl (x=o,m,p), x-(trifluoromethyl)phenyl (x=o,m,p), x-(trifluoromethoxy)phenyl (x=o,m,p), x-(trifluoromethylsulfonyl)phenyl (x=o,m,p), x-fluorophenyl (x=o,m,p), x-chlorophenyl (x=o,m,p), x-bromophenyl (x=o,m,p), x-iodophenyl (x=o,m,p), x,y-dimethylphenyl (x=2,3; 2,4; 2,5; 2,6; 3,4; 3,5), x,y-dihydroxyphenyl (x=2,3; 2,4; 2,5; 2,6; 3,4; 3,5), x,y-difluorophenyl (x=2,3; 2,4; 2,5; 2,6; 3,4; 3,5), x,y-dichlorophenyl (x=2,3; 2,4; 2,5; 2,6; 3,4; 3,5), x,y-dibromophenyl (x=2,3; 2,4; 2,5; 2,6; 3,4; 3,5), x,y-dimethoxyphenyl (x=2,3; 2,4; 2,5; 2,6; 3,4; 3,5), 5-fluoro-2-methylphenyl, 3,4,5-trimethoxyphenyl and 2,4,5-trimethylphenyl;
an unsubstituted, mono- or polysubstituted C₆-C₁₅-aryl group here includes, the C₆-C₁₀-aryl group and anthryl, phenanthryl, phenalenyl; an unsubstituted, mono- or polysubstituted C₆-C₂₀-aryl group here includes, the C₆-C₁₅-aryl group and pyrenyl, chrysenyl naphthacenyl, picenyl;
where furthermore Hal, Alk¹, Arl¹, Het¹, OH, OAlk¹, OArl¹, O Het¹, NH₂, NHAlk¹, NAlk¹₂, NHArl¹, NArl¹₂, NHHet¹, NHet¹₂, CN, COOH, COHal, CONHAlk¹, CONAlk¹₂, COOAlk¹, CONHArl¹, CONArl¹₂, COOArl¹, CONHHet¹, CONHet¹₂, COOHet¹, SO₂OH, SO₂Hal, SO₂CF₃, SO₂OAlk¹, SO₂NHAlk¹, SO₂NAlk¹₂, SO₂OArl¹, SO₂NHArl¹, SO₂NArl¹₂, SO₂OHet¹, SO₂NHHet¹, SO₂NHet¹₂, SCF₃, and NO₂ are included as substituents on the respective aryl group;
an unsubstituted, mono- or polysubstituted C₅-C₆-heteroaryl group here includes, x-furyl (x=2,3), x-thienyl (x=2,3), x-pyrrolyl (x=1,2,3), x-imidazolyl (x=1,2,4,5), x-pyrazolyl (x=3,4,5), x-oxazolyl (x=2,4,5), x-isoxazolyl (x=3,4,5), x-thiazolyl (x=2,4,5), x-isothiazolyl (x=3,4,5), x-pyridyl (x=2,3,4), x-pyrimidinyl (x=2,4,5,6), x-pyrazinyl (x=2,3,5,6), x-pyridazinyl (x=3,4,5,6), 1,2,3-triazol-x-yl (x=1,4,5), 1,2,4-triazol-x-yl (x=1,3,5), x-tetrazolyl (x=1,5), 1,2,3-oxadiazol-x-yl (x=4,5), 1,2,4-oxadiazol-x-yl (x=3,5), 1,3,4-thiadiazol-x-yl (x=2,5), 1,2,4-thiadiazol-x-yl (x=3,5), 1,2,3-thiadiazol-x-yl (x=4,5), x-2H-thiopyranyl (x=2,3,4,5,6), x-4H-thiopyranyl (x=2,3,4) and x-pyrrolidinyl (x=1,2,3);
an unsubstituted, mono- or polysubstituted C₆-C₁₀-heteroaryl group here includes, the C₆-C₁₀-heteroaryl group and x-benzofuryl (x=2,3,4,5,6,7), x-benzothienyl (x=2,3,4,5,6,7), x-1H-indolyl (x=1,2,3,4,5,6,7), x-benzimidazolyl (x=1,2,4,5), x-benzopyrazolyl (x=1,3,4,5,6,7), x-benzoxazolyl (x=2,4,5,6,7), x-benzisoxazolyl (x=3,4,5,6,7), x-benzothiazolyl (x=2,4,5,6,7), x-benzisothiazolyl (x=2,4,5,6,7), x-benz-1,2,3-oxadiazolyl (x=4,5,6,7), x-quinolinyl (x=1,2,3,4,5,6,7,8), x-isoquinolinyl (x=1,3,4,5,6,7,8), x-quinazolinyl (x=2,4,5,6,7,8) and x-benzo-1,2,3-triazolxl (x=1,6,7,8,9);
an unsubstituted, mono- or polysubstituted C₆-C₂₀-heteroaryl group here includes, the C₆-C₁₀-heteroaryl group and
x-carbazolyl (x=1,2,3,4,9), x-acridinyl (x=1,2,3,4,5,6,7,8,9) and x-cinnolinyl (x=3,4,5,6,7,8);
where Hal, Alk¹, Arl¹, Het¹, OH, OAlk¹, OArl¹, 0 Het¹, NH₂, NHAlk¹, NAlk¹₂, NHArl¹, NArl¹₂, NHHet¹, NHet¹₂, CN, COOH, COHal, CONHAlk¹, CONAlk¹₂, COOAlk¹, CONHArl¹, CONArl¹₂, COOArl¹, CONHHet¹, CONHet¹₂, COOHet¹, SO₂OH, SO₂Hal, SO₂CF₃, SO₂OAlk¹, SO₂NHAlk¹, SO₂NAlk¹₂, SO₂OArl¹, SO₂NHArl¹, SO₂NArl¹₂, SO₂OHet¹, SO₂NHHet¹, SO₂NHet¹₂, SCF₃, and NO₂ are included as substituents on the respective heteroaryl group

Alk³ therefore, besides the straight-chain or branched alkyl groups having 1 to 12 C atoms listed above, also includes, in particular, alkyl groups of the formula —CH₂—CH(OH)—CH₂—O-Alk⁴, where Alk⁴ may have one of the meanings indicated above. Alk⁴ in the formula —CH₂—CH(OH)—CH₂—O-Alk⁴ indicated is preferably isopropyl.

The invention thus relates to the use of a compound of the formula I

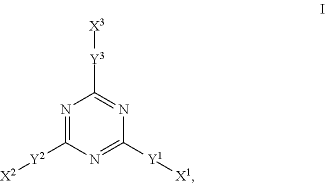

where Y¹, Y² and Y³ each stand, independently of one another, for a single bond or NH,
where X¹, X² and X³ each stand, independently of one another, for Alk¹ or a substituent of the formula II or III

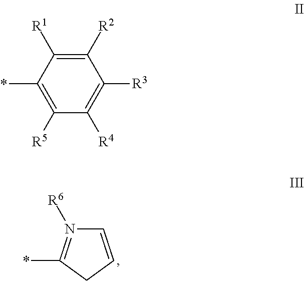

where R¹ to R⁵ each stand, independently of one another,
for H, OH, Hal, Alk¹, OAlk¹, SAlk¹, NHAlk¹, N(Alk¹)₂, COOAlk¹, COOH, C(O)H, CONHAlk¹, CONH₂, COO⁻Kt⁺, Cyc¹, OCyc¹, Arl¹, OArl¹, COOArl¹, biphenylyl, Het¹, OHet¹, Si(Alk²)₃, OEth, COOEth or for a substituent of the formula IV

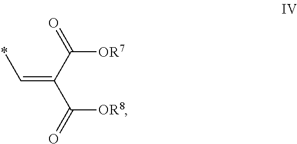

where Alk¹ in each case stands, independently of one another,
for a straight-chain or branched C₁- to C₂₀-alkyl group or for a straight-chain or branched C₂- to C₂₀-alkenyl group, which has at least one double bond, or for a straight-chain or branched C₂- to C₂₀-alkynyl group, which has at least one triple bond, and/or in which at least one or more non-adjacent C atoms of the alkyl, alkenyl or alkynyl group may be replaced by O or trimethylsilyl and/or which may contain at least one OH bonded to a primary or secondary C atom,
where Cyc¹ in each case stands, independently of one another, for a C₃- to C₈-cycloalkyl group, which may have at least one double bond, and/or in which at least one CH₂ may be replaced by O or NH,
where Arl¹ in each case stands, independently of one another, for an unsubstituted, mono- or polysubstituted C₆- to C₂₀-aryl group,
where Het¹ in each case stands, independently of one another, for an unsubstituted, mono- or polysubstituted C₅- to C₂₀-aryl group, in which at least one CH₂ has been replaced by O, S or NH,
where Alk² in each case stands, independently of one another, for a straight-chain or branched C₁- to C₁₂-alkyl group, which may be interrupted by at least one O,
where R⁶ stands for Alk¹,
where R⁷ and R⁸ each stand, independently of one another, for Alk¹ or Arl¹,
where Eth¹ in each case stands, independently of one another, for —(CH₂—CH₂—O)ₘ₁-Alk² where 1≦m1≦16,
where Hal stands for F, Cl, Br or I,
where Kt⁺ stands for Li⁺, Na⁺ or K⁺,
and/or salt thereof as accelerator for a photoinduced reaction of 3-(4-tert-butylphenyl)-3-hydroxy-1-(4-methoxyphenyl)propan-1-one, 1-(4-tert-butyl-phenyl)-3-hydroxy-3-(4-methoxyphenyl)propan-1-one or mixtures of these compounds to give avobenzone.

Advantageously, the use of at least one compound of the formula I enables the loss of UV absorption capacity of a composition or material to be at least partially compensated owing to acceleration of the photoinduced reaction of 3-(4-tert-butylphenyl)-3-hydroxy-1-(4-methoxyphenyl)-propan-1-one, 1-(4-tert-butylphenyl)-3-hydroxy-3-(4-methoxyphenyl)propan-1-one or the mixture of the two compounds to give avobenzone, since avobenzone and thus a known UV filter is formed owing to the photo-induced reaction on irradiation.

Owing to the use of at least one compound of the formula I as accelerator for the photoinduced reaction, virtually complete conversion of 3-(4-tert-butylphenyl)-3-hydroxy-1-(4-methoxyphenyl)propan-1-one, 1-(4-tert-butyl-phenyl)-3-hydroxy-3-(4-methoxyphenyl)propan-1-one or the mixtures of these compounds into avobenzone is also possible within a predetermined period.

This predetermined period t is preferably selected from the time interval which corresponds to a minimal erythemal dose (MED) of 0.5 to 50 MED, particularly preferably 5.0 to 30 MED and very particularly preferably 10 to 20 MED. An absolute UV dose of 50 kJ/m² applied by solar radiation approximately corresponds here to the erythema-weighted dose of 1 MED.

The use of 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or the mixtures of these compounds and at least one compound of the formula I thus facilitates variable sun protection which liberates more and more of the UV filter avobenzone on increasing irradiation duration through photoconversion of 3-(4-tert-butylphenyl)-3-hydroxy-1-(4-methoxyphenyl)-propan-1-one, 1-(4-tert-butylphenyl)-3-hydroxy-3-(4-methoxyphenyl)propan-1-one or the mixtures of these compounds, in particular of 3-(4-tert-butylphenyl)-3-hydroxy-1-(4-methoxyphenyl)propan-1-one.

3-(4-tert-Butylphenyl)-3-hydroxy-1-(4-methoxyphenyl)propan-1-one, 1-(4-tert-butylphenyl)-3-hydroxy-3-(4-methoxyphenyl)propan-1-one or the mixtures of these compounds together with at least one compound of the formula I, as described above, is preferably used in combination with at least one further UV filter. The corresponding UV filter added in combination preferably has an absorption spectrum which is the same as the absorption spectrum of avobenzone, at least with respect to the absorption maximum of avobenzone. The UV filter used in combination is particularly preferably avobenzone itself. It is very particularly preferred if the UV filter added in combination is different from avobenzone and is additionally photostable. In this arrangement, no avobenzone is initially present before commencement of the irradiation. However, additional absorption power compared with the initial value is built up here by photoconversion. Examples of UV filters of this type which are added in combination and are marketed as photostable are, for example, hexyl 2-(4-diethylamino-2-hydroxy-benzoyl)benzoate, marketed by BASF SE under the trade names Uvinul® A Plus, 2,2′-methylene-bis-(6-(2H-benzotriazol-2-yl)-4-(1,1,3,3-tetramethylbutyl)phenol), marketed by BASF SE under the name Tinosorb® M, or salts of 2,2′-(1,4-phenylene)bis)-1H-benzimidazole-4,6-disulfonic acid), for example marketed by Symrise under the name Neo Heliopan® AP, 2-(2H-benzotriazol-2-yl)-4-methyl-6-(2-methyl-3-(1,3,3,3-tetramethyl-1-(trimethyl-silyloxy)disiloxanyl)propyl)phenol (INCI Drometriazole Trisiloxane), for example known under the trade name Mexoryl XL from I'Oréal, or 3,3′-(1,4-phenylenedimethylene)bis(7,7-dimethyl-2-oxobicyclo-[2.2.1]hept-1-yl-methanesulfonic acid (INCI Terephthalidene Dicamphor Sulfonic Acid), for example known under the trade name Mexoryl SX from I'Oréal.

Thus, the use of 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or the mixtures of these compounds, in particular of 3-(4-tert-butyl-phenyl)-3-hydroxy-1-(4-methoxyphenyl)propan-1-one, together with at least one compound of the formula I, as described above, enables either the photoinduced decomposition of a UV filter of this type added in combination to be at least partially compensated with respect to the reducing UV absorption capacity by means of the formation of avobenzone, or where appropriate even enables additional absorption power to be generated.

Preference is given to the use of a mixture of at least one compound of the formula I together with 3-(4-tert-butylphenyl)-3-hydroxy-1-(4-methoxyphenyl)propan-1-one, 1-(4-tert-butylphenyl)-3-hydroxy-3-(4-methoxy-phenyl)propan-1-one or the mixtures of these compounds, in particular of 3-(4-tert-butylphenyl)-3-hydroxy-1-(4-methoxyphenyl)propan-1-one, as photoinduced stabiliser for a UV filter. The UV filter to be stabilised particularly preferably has an energy level of a first excited triplet state which corresponds to the energy level of the first excited triplet state of 3-(4-tert-butylphenyl)-3-hydroxy-1-(4-methoxyphenyl)propan-1-one or 1-(4-tert-butylphenyl)-3-hydroxy-3-(4-methoxyphenyl)propan-1-one with a deviation of a maximum of five percent. The mixture described above is particularly preferably used as photoinduced stabiliser for avobenzone.

Irradiation of molecules with UV rays enables them to absorb the radiation energy. The molecules here are usually raised from a ground state S₀ into an excited first singlet state S₁ or a higher singlet state S. The molecules can change from a singlet state of this type into a triplet state by intersystem crossing (ISC), and from there fall back into the ground state by, for example, thermal relaxation or radiation relaxation. It is thus possible for a molecule of this type to be excited from a ground state S₀ into a first singlet state S₁ by absorption of UV rays, where it subsequently changes into the first excited triplet state T₁ by intersystem crossing (ISC) and can then fall back into the ground state S₀ again by relaxation processes.

A mixture of at least one compound of the formula I together with 3-(4-tert-butylphenyl)-3-hydroxy-1-(4-methoxyphenyl)propan-1-one, 1-(4-tert-butyl-phenyl)-3-hydroxy-3-(4-methoxyphenyl)propan-1-one or the mixtures of these compounds, in particular of 3-(4-tert-butylphenyl)-3-hydroxy-1-(4-methoxyphenyl)propan-1-one, and at least one further UV filter is preferably used as UV filter system for a composition, where the composition comprising a UV filter system of this type is distinguished by essentially constant or increasing UV absorption capacity in a predetermined period. Examples of a further UV filter, besides the at least one compound of the formula I, as described above, are listed below.

Constant or increasing UV absorption capacity of this type of a UV filter system of this type enables sun protection which ensures protection of the skin or the respective material provided with a UV filter system of this type which ensures at least constant UV absorption capacity over the predetermined period.

Particularly in the case of the skin, increasing UV absorption capacity is particularly desired, since the skin is increasingly irritated with progressive exposure thereof to the radiation. The skin thus also requires greater protection with progressive exposure to radiation, which is advantageously facilitated by sun protection of this type with a UV filter system of this type. A UV filter system of this type is capable of intensifying the protection of the skin with progressive irritation. Thus, the skin receives the protection that it requires compared with conventional sun-protection compositions, which generally exhibit reducing UV absorption capacity with progressive exposure to radiation.

A further central idea of the invention is the use of a mixture of at least one compound of the formula I, as described above, together with 3-(4-tert-butylphenyl)-3-hydroxy-1-(4-methoxyphenyl)propan-1-one, 1-(4-tert-butyl-phenyl)-3-hydroxy-3-(4-methoxyphenyl)propan-1-one or the mixtures of these compounds, in particular of 3-(4-tert-butylphenyl)-3-hydroxy-1-(4-methoxyphenyl)propan-1-one, as photoinduced-antioxidative system. This is, in particular, a non-therapeutic use.

The photoinduced reaction of 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or the mixtures of these compounds, in particular of 3-(4-tert-butylphenyl)-3-hydroxy-1-(4-methoxyphenyl)propan-1-one, to give avobenzone is distinguished, purely formally, by the fact that hydrogen equivalents are released in the course of the conversion of one of the compounds, as described above, into avobenzone. The hydrogen equivalents are generally not liberated, but instead are taken up by surrounding compounds. A mixture of 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or the mixtures of these compounds, in particular of 3-(4-tert-butylphenyl)-3-hydroxy-1-(4-methoxyphenyl)propan-1-one, and avobenzone therefore has an antioxidative and thus photoantioxidative action on irradiation.

A photoinduced-antioxidative system is thus taken to mean a mixture of 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or the mixtures of these compounds, in particular of 3-(4-tert-butylphenyl)-3-hydroxy-1-(4-methoxyphenyl)propan-1-one, and at least one compound of the formula I, as described above, which exhibits an antioxidative action on irradiation.

The invention furthermore relates to a composition comprising at least one vehicle which is suitable for cosmetic, pharmaceutical, dermatological compositions, foods, food supplements, household products or plastics, and a mixture of at least one compound of the formula I, as described above, and 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or the mixtures of these compounds, in particular of 3-(4-tert-butylphenyl)-3-hydroxy-1-(4-methoxyphenyl)propan-1-one, and optionally a further UV filter. The optionally further UV filter or in other words the UV filter added in combination particularly preferably has an absorption spectrum which is the same as the absorption spectrum of avobenzone with respect to the absorption maximum. It is very particularly preferred for the UV filter added in combination to be different from avobenzone and in addition to be photostable, as described above. Examples of UV filters of this type which are marketed as photostable and are added in combination are, for example, hexyl 2-(4-diethylamino-2-hydroxybenzoyl)benzoate, marketed by BASF SE under the trade names Uvinul® A Plus, 2,2′-methylene-bis-(6-(2H-benzotriazol-2-yl)-4-(1,1,3,3-tetramethylbutyl)phenol), marketed by BASF SE under the name Tinosorb® M, or salts of 2,2′-(1,4-phenylene)-bis)-1H-benzimidazole-4,6-disulfonic acid), for example marketed by Symrise under the name Neo Heliopan® AP, 2-(2H-benzotriazol-2-yl)-4-methyl-6-(2-methyl-3-(1,3,3,3-tetramethyl-1-(trimethylsilyloxy)disiloxanyl)propyl)phenol (INCI Drometriazole Trisiloxane), for example known under the trade name Mexoryl XL from I'Oréal, or 3,3′-(1,4-phenylenedimethylene)-bis(7,7-dimethyl-2-oxobicyclo-[2.2.1]hept-1-ylmethanesulfonic acid (INCI Terephthalidene Dicamphor Sulfonic Acid), for example known under the trade name Mexoryl SX from I'Oréal.

In order that the number of UV filters in a composition can be kept small, it is particularly preferred in an embodiment for the UV filter added in combination in the mixture according to the invention to be avobenzone.

A composition according to the invention, as described above, preferably has at most a UV absorption capacity differing by +/−5% during irradiation with sunlight or sun-like light within a predetermined period.

Sun-like light is taken to mean radiation in a spectral composition analogous to solar radiation or sunlight. Sun-like light of this type can be generated, for example, by a solar simulator or used in solariums, where the spectral composition of the solarium radiation is not exactly the same as that of sunlight. Accordingly, sun-like light is also intended to be taken to mean light which is at least partly equal to the spectral composition of sunlight or which develops a sunlight-like action.

A composition of this type particularly preferably has increasing UV absorption capacity during irradiation with sunlight or sun-like light within a predetermined period.

The invention furthermore relates to a method for the stabilisation of the UV absorption capacity of a composition by means of a mixture of at least one compound of the formula I, as described above, together with 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or the mixtures of these compounds, in particular together with 3-(4-tert-butylphenyl)-3-hydroxy-1-(4-methoxyphenyl)propan-1-one, where the mixture and the composition are matched to one another in such a way that the UV absorption capacity of the composition has a maximum deviation of +/−5 percent or increases in a predetermined period on irradiation with sunlight or sun-like light.

Preferred compounds of the formula I which are used in accordance with the invention are compounds of the formula I in which X¹ and X² each stand, independently of one another, for a substituent of the formula II, and X³ stands for Alk¹ or for a substituent of the formula II.

In the case of the substituents of the formula II, it is preferred for R² and R⁵ to denote H.

In the case of the substituents of the formula II, it is preferred for R¹, R³ and R⁴ each to stand, independently of one another, for H, OH, Alk¹, OAlk¹, Arl¹, biphenylyl, COOAlk¹, CONHAlk¹, Het¹ or a substituent of the formula IV

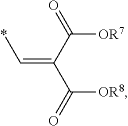

where R⁷ and R⁸ have a meaning indicated above.

If X³ stands for Alk¹, it is preferred for Alk¹ to denote Alk² or particularly preferably to denote Alk⁴. Alk¹ in the definition of X³ is very particularly preferably 2-ethylhexyl.

In the case of the respectively independent substituents R¹, R³ and R⁴ in the formula II, it is preferred for Alk¹ to correspond to the definition of Alk² or Alk⁴ or it is very particularly preferred for Alk¹ to denote methyl or 1,1-dimethylpropyl.

In the case of the respectively independent substituents R¹, R³ and R⁴ in the formula II, it is preferred for OAlk¹ to correspond to OAlk³ or it is very particularly preferred for OAlk¹ to denote methoxy, 2-ethylhexyloxy or O—CH₂—CH(OH)—CH₂—O-isopropyl.

In the case of the respectively independent substituents R¹, R³ and R⁴ in the formula II, it is preferred for Arl¹ to denote unsubstituted phenyl.

In the case of the respectively independent substituents R¹, R³ and R⁴ in the formula II, it is preferred for COOAlk¹ to denote COOAlk² or it is very particularly preferred for COOAlk¹ to denote 2-ethylhexylcarboxy.

In the case of the respectively independent substituents R¹, R³ and R⁴ in the formula II, it is preferred for CONHAlk¹ to denote CONHAlk² or it is very particularly preferred for CONHAlk¹ to denote tert-butylaminocarboxy.

In the case of the respectively independent substituents R¹, R³ and R⁴ in the formula II, it is preferred for the substituents R⁷ and R⁸ in formula IV to correspond to Alk⁴ or it is very particularly preferred for R⁷ and R⁸ to correspond, independently of one another, to ethyl, isobutyl or isopropyl. R⁷ and R⁸ are particularly preferably identical.

In the case of the respectively independent substituents R¹, R³ and R⁴ in the formula II, it is preferred for Het¹ preferably to denote

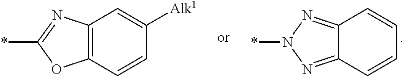

Het¹ is very particularly preferably

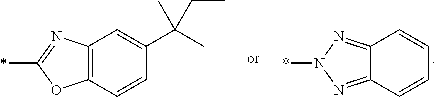 [SMILES: *c1nc2cc(C(C)(C)CC)ccc2o1 ; *n1nc2ccccc2n1]

Particularly preferred compounds of the formula I are compounds in which Y³—X³ is selected from the group of the combinations
a) Y³ stands for NH and X³ stands for Alk¹ or for a particularly preferred meaning above for Alk¹,
b) Y³ stands for a single bond and X³ stands for a substituent of the formula II, where R¹, R², R⁴ and R⁵ denote H and R³ denotes OAlk¹, Arl¹ or biphenylyl and where OAlk¹, Arl¹ may have one of the preferred meanings given above or
c) Y³ stands for NH and X³ stands for a substituent of the formula II, where R¹ denotes H or Alk¹, R² and R⁵ denote H, R⁴ denotes H or OH and R³ denotes COOAlk¹, CONHAlk¹, Het¹ or a substituent of the formula IV, where Alk¹, COOAlk¹, CONHAlk¹, Het¹ or the substituent of the formula IV with R⁷ and R⁸ may have one of the meanings indicated above.

Particularly preferred compounds of the formula I are compounds in which Y¹—X¹ and Y²—X² are each selected, independently of one another, from the group of the combinations
d) Y¹ and Y² stand for a single bond and X¹ and X² each stand, independently of one another, for a substituent of the formula II, where R¹ denotes H, OAlk¹ or OH, R², R⁴ and R⁵ denote H and R³ denotes OAlk¹, Arl¹, Alk¹ or biphenylyl and where Alk¹, OAlk¹, Arl¹ may have one of the preferred meanings given above or
e) Y¹ and Y² stand for NH and X¹ and X² each stand, independently of one another, for a substituent of the formula II, where R¹ denotes H or OH, R² and R⁵ denote H, R⁴ denotes H or Alk¹ and R³ denotes COOAlk¹, Het¹ or a substituent of the formula IV, where Alk¹, COOAlk¹, Het¹ or the substituent of the formula IV with R⁷ and R⁸ may have one of the meanings indicated above.

Y¹—X¹ and Y²—X² are particularly preferably identical.

Further preferred combinations of embodiments are disclosed in the claims.

Very particular preference is given in accordance with the invention to compounds of the formulae X, XI, XII, XIII, XIV, XV, XVI, XVII, XVIII, XIX, XX, XXI or XXII

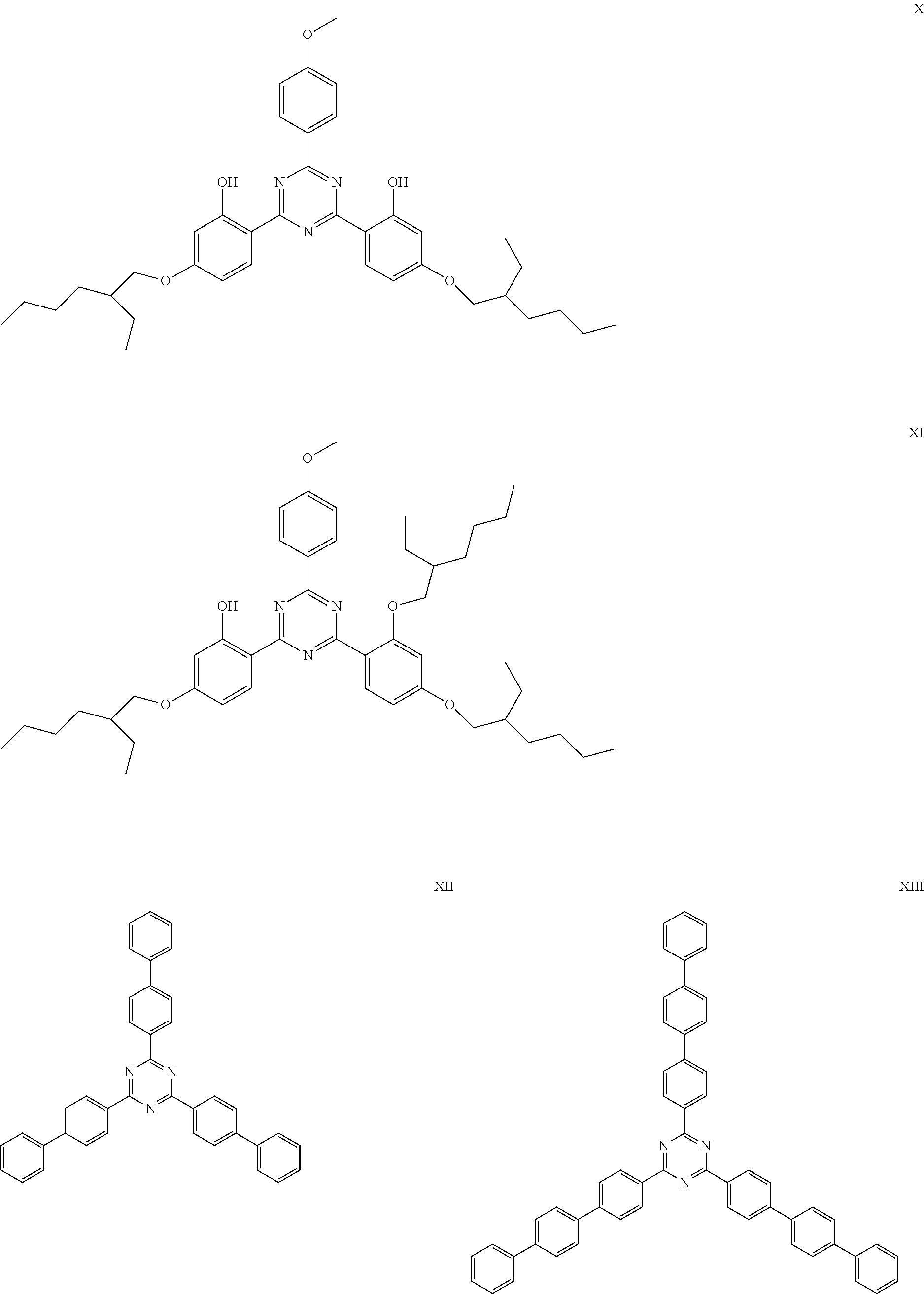

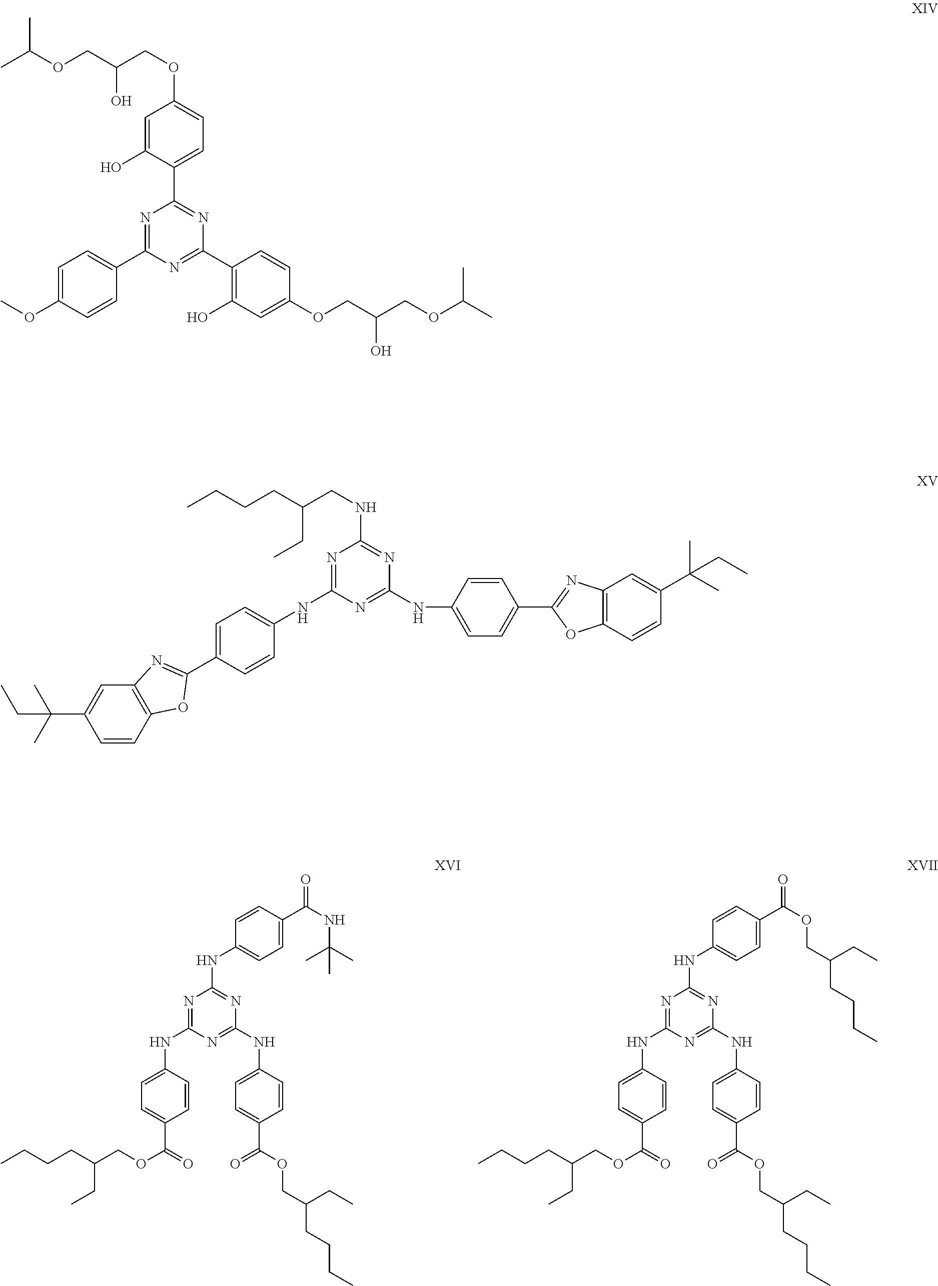

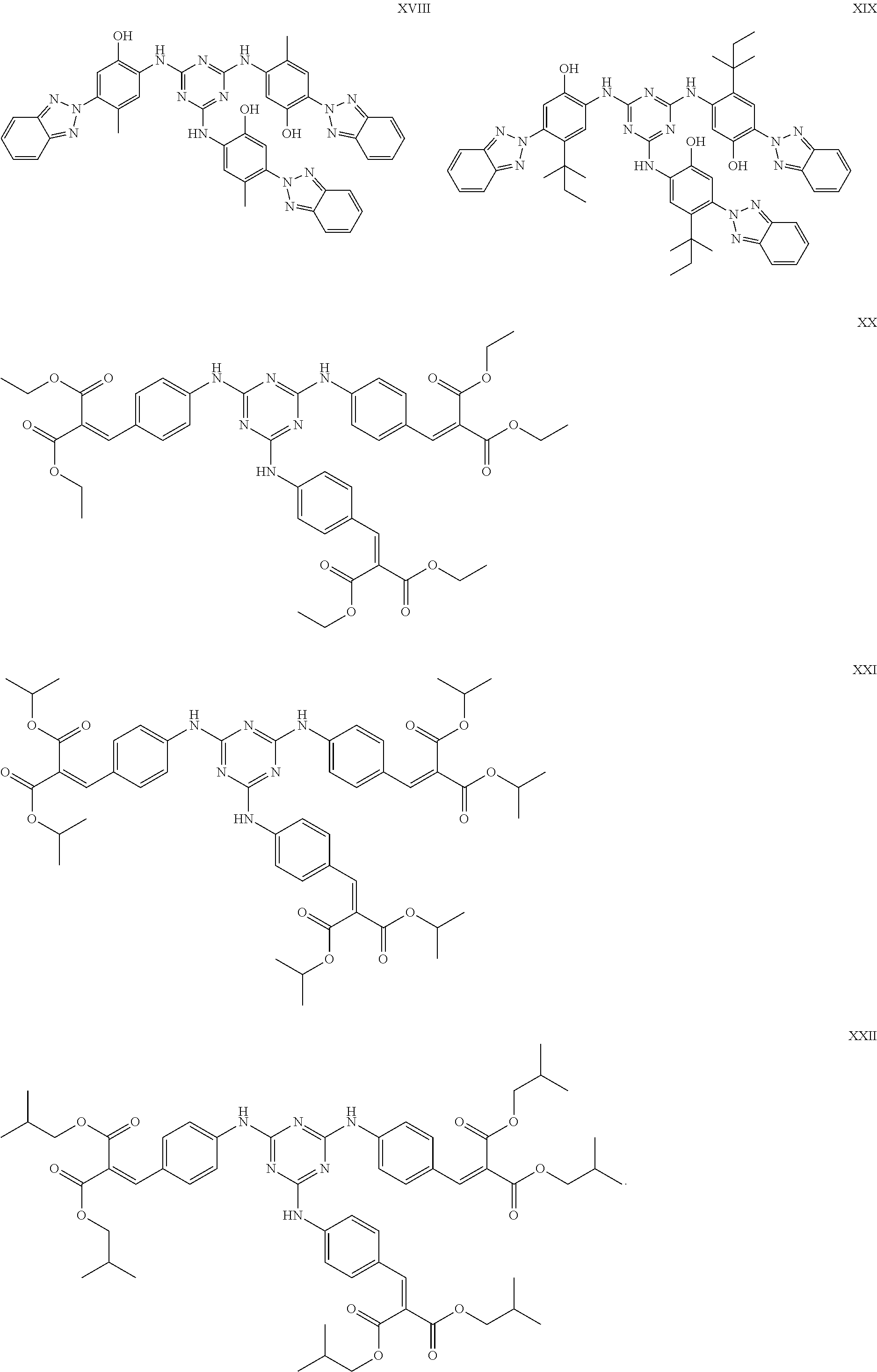

From this group of individual compounds as representative of the compounds of the formula I, the individual compounds of the formula X, XII, XVI, XVII and XV are particularly preferred in the use according to the invention and should particularly preferably be used in the compositions and mixtures according to the invention. The compound of the formula XVII is very particularly preferably selected.

Compounds of the formula I which are bonded to a polysiloxane by at least one X¹ to X³ should likewise be used in accordance with the invention.

Triazines of this type are described in WO 2009/074409, in WO 2009/053149 and in WO 2006/128732 and are hereby disclosed in accordance with the invention.

The present invention furthermore relates to compositions which comprise 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or mixtures of these compounds, in particular of 3-(4-tert-butylphenyl)-3-hydroxy-1-(4-methoxyphenyl)propan-1-one, with at least one compound of the formula I or with at least one of the compounds of the formula I described above or indicated as preferred or the individual compounds listed.

The compositions here are usually compositions which can be applied topically, for example cosmetic or dermatological formulations or medical products. In this case, the compositions comprise a cosmetically or dermatologically suitable vehicle and, depending on the desired property profile, optionally further suitable ingredients. In the case of pharmaceutical compositions, the compositions in this case comprise a pharmaceutically tolerated vehicle and optionally further pharmaceutical active compounds.

“Can be applied topically” in the sense of the invention means that the composition is applied externally and locally, i.e. that the composition must be suitable for, for example, application to the skin. Preferred compositions are cosmetic compositions.

In the sense of the present invention, the term agent or formulation is also used synonymously alongside the term composition.

The compositions may include or comprise, essentially consist of or consist of the said requisite or optional constituents. All compounds or components which can be used in the compositions are either known and commercially available or can be synthesised by known processes.

The invention also relates to a process for the preparation of a composition, as described above, in which at least one compound of the formula I is mixed together with 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or mixtures of these compounds, in particular with 3-(4-tert-butylphenyl)-3-hydroxy-1-(4-methoxyphenyl)propan-1-one, and with a vehicle and optionally with further active compounds or assistants. Suitable vehicles and further UV filters or active compounds or assistants are described in detail in the following part.

In preferred embodiments, the at least one compound of the formula I having the substituents which are defined or indicated as preferred or preferred individual compounds is typically employed in the compositions according to the invention in amounts of 0.05 to 10% by weight, preferably in amounts of 0.1% by weight to 5% by weight and particularly preferably in amounts of 0.5 to 2% by weight. The person skilled in the art is presented with absolutely no difficulties here in selecting the amounts correspondingly depending on the intended action of the composition.

In the compositions described which, in accordance with the invention, comprise at least one compound of the formula I with the individual compounds defined or indicated as preferred and 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or mixtures of these compounds, in particular 3-(4-tert-butylphenyl)-3-hydroxy-1-(4-methoxyphenyl)-propan-1-one, coloured pigments may furthermore also be present, where the layer structure of the pigments is not limited.

The coloured pigment should preferably be skin-coloured or brownish on use of 0.1 to 5% by weight. The choice of a corresponding pigment is familiar to the person skilled in the art.

Besides the compounds of the formula I, as described above or described as preferred, preferred compositions comprise further UV filters, for example organic UV filters. These are, for example, the so-called hydrophilic or lipophilic sun-protection filters, which are effective in the UVA region and/or UVB region and(/or IR and/or VIS region (absorbers). These substances can be selected, in particular, from cinnamic acid derivatives, salicylic acid derivatives, camphor derivatives, triazine derivatives having a different structure than the compound of the formula I employed in the composition, β,β-diphenylacrylate derivatives, p-aminobenzoic acid derivatives and polymeric filters and silicone filters, which are described in the application WO-93/04665. Further examples of organic filters are indicated in the patent application EP-A 0 487 404. The said UV filters are usually named below in accordance with INCI nomenclature.

Particularly suitable for a combination are:

para-aminobenzoic acid and derivatives thereof: PABA, Ethyl PABA, Ethyl dihydroxypropyl PABA, Ethylhexyl dimethyl PABA, for example marketed by ISP under the name “Escalol 507”, Glyceryl PABA, PEG-25 PABA, for example marketed under the name “Uvinul P25” by BASF.

Salicylates: Homosalate marketed by Merck under the name “Eusolex HMS”; Ethylhexyl salicylate, for example marketed by Symrise under the name “Neo Heliopan OS”, Dipropylene glycol salicylate, for example marketed by Scher under the name “Dipsal”, TEA salicylate, for example marketed by Symrise under the name “Neo Heliopan TS”.

β,β-Diphenylacrylate derivatives: Octocrylene, for example marketed by Merck under the name “Eusolex® OCR”, “Uvinul N539” from BASF, Etocrylene, for example marketed by BASF under the name “Uvinul N35”.

Benzophenone derivatives: Benzophenone-1, for example marketed under the name “Uvinul 400”; Benzophenone-2, for example marketed under the name “Uvinul D50”; Benzophenone-3 or Oxybenzone, for example marketed under the name “Uvinul M40”; Benzophenone-4, for example marketed under the name “Uvinul MS40”; Benzophenone-9, for example marketed by BASF under the name “Uvinul DS-49”, Benzophenone-5, Benzophenone-6, for example marketed by Norquay under the name “Helisorb 11”, Benzophenone-8, for example marketed by American Cyanamid under the name “Spectra-Sorb UV-24”, Benzophenone-12 n-hexyl 2-(4-diethylamino-2-hydroxybenzoyl)benzoate or 2-hydroxy-4-methoxybenzophenone, marketed by Merck, Darmstadt, under the name Eusolex® 4360.

Benzylidenecamphor derivatives: 3-Benzylidenecamphor, for example marketed by Chimex under the name “Mexoryl SD”, 4-Methylbenzylidene-camphor, for example marketed by Merck under the name “Eusolex 6300”, benzylidenecamphorsulfonic acid, for example marketed by Chimex under the name “Mexoryl SL”, Camphor benzalkonium methosulfate, for example marketed by Chimex under the name “Mexoryl SO”, terephthalylidene-dicamphorsulfonic acid, for example marketed by Chimex under the name “Mexoryl SX”, Polyacrylamidomethylbenzylidenecamphor marketed by Chimex under the name “Mexoryl SW”.

Phenylbenzimidazole derivatives: phenylbenzimidazolesulfonic acid, for example marketed by Merck under the name “Eusolex 232”, disodium phenyl bibenzimidazole tetrasulfonate, for example marketed by Symrise under the name “Neo Heliopan AP”.

Phenylbenzotriazole derivatives: Drometrizole trisiloxane, for example marketed by Rhodia Chimie under the name “Silatrizole”, Methylenebis(benzotriazolyl)tetramethylbutylphenol in solid form, for example marketed by Fairmount Chemical under the name “MIXXIM BB/100”, or in micronised form as an aqueous dispersion, for example marketed by the former BASF under the name “Tinosorb M”.

Anthraniline derivatives: Menthyl anthranilate, for example marketed by Symrise under the name “Neo Heliopan MA”.

Imidazole derivatives: Ethylhexyldimethoxybenzylidenedioxoimidazoline propionate.

Benzalmalonate derivatives: polyorganosiloxanes containing functional benzalmalonate groups, such as, for example, polysilicone-15, for example marketed by Hoffmann LaRoche under the name “Parsol SLX”.

4,4-Diarylbutadiene derivatives: 1,1-Dicarboxy(2,2′-dimethylpropyl)-4,4-diphenylbutadiene.

Benzoxazole derivatives: 2,4-bis[5-(1-dimethylpropyl)benzoxazol-2-yl(4-phenyl) imino]-6-(2-ethylhexyl)imino-1,3,5-triazine, for example marketed by Sigma 3V under the name Uvasorb K2A, and mixtures comprising this.

Piperazine derivatives, such as, for example, the compound

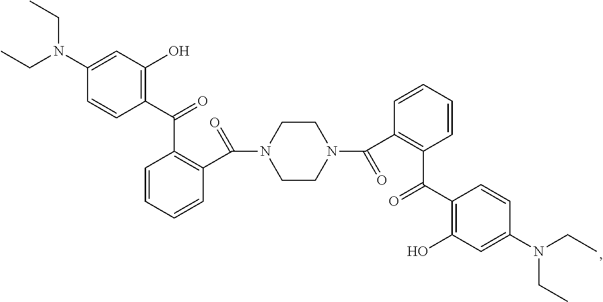 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

or the UV filters of the following structures

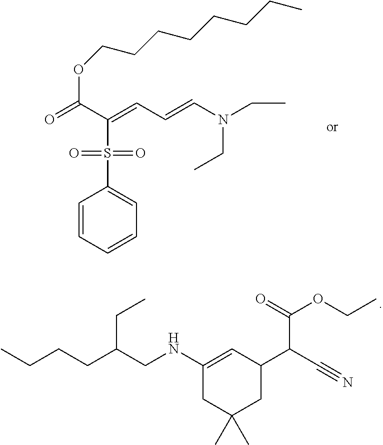

Preferably can also be combined with UV filters based on polysiloxane copolymers having a random distribution in accordance with the following formula, where, for example, a=1.2; b=58 and c=2.8:

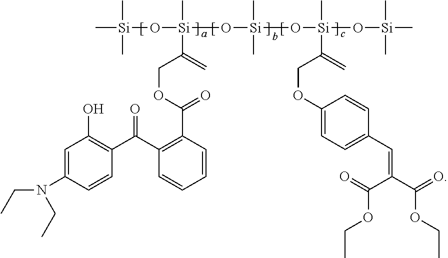

The compounds listed should only be regarded as examples. It is of course also possible to use other UV filters.

Suitable organic UV-protecting substances which are suitable as further UV filters can preferably be selected from the following list: Ethylhexyl salicylate, Phenylbenzimidazolesulfonic acid, Benzophenone-3, Benzophenone-4, Benzophenone-5, n-Hexyl 2-(4-diethylamino-2-hydroxy-benzoyl)benzoate, 4-Methylbenzylidenecamphor, Terephthalylidene-dicamphorsulfonic acid, Disodium phenyldibenzimidazoletetrasulfonate, Methylenebis(benzotriazolyl)tetramethylbutylphenol, Ethylhexyl Triazone, Diethylhexyl Butamido Triazone, Drometrizole trisiloxane, Polysilicone-15, 1,1-Dicarboxy(2,2′-dimethylpropyl)-4,4-diphenylbutadiene, and mixtures thereof. Very particularly preferred organic UV filters which can be combined in accordance with the invention with the compounds of the formula I and 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or mixtures of these compounds, in particular 3-(4-tert-butylphenyl)-3-hydroxy-1-(4-methoxyphenyl)propan-1-one, have been described above.

These organic UV filters are generally incorporated into formulations in an amount of 0.01 percent by weight to 20 percent by weight, preferably 1% by weight—10% by weight.

Besides the compounds of the formula I and 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or mixtures of these compounds, in particular 3-(4-tert-butylphenyl)-3-hydroxy-1-(4-methoxyphenyl)-propan-1-one, compositions according to the invention may comprise further inorganic UV filters, so-called particulate UV filters.

These combinations with particulate UV filters are possible both as powder and also as dispersion or paste of the following types.

Preference is given here both to those from the group of the titanium dioxides, such as, for example, coated titanium dioxide (for example Eusolex®T-2000, Eusolex®T-AQUA, Eusolex®T-AVO, Eusolex®T-OLEO), zinc oxides (for example Sachtotec®), iron oxides or also cerium oxides and/or zirconium oxides.

Furthermore, combinations with pigmentary titanium dioxide or zinc oxide are also possible, where the particle size of these pigments are greater than or equal to 200 nm, for example Hombitan® FG or Hombitan® FF-Pharma.

It may furthermore be preferred for the compositions to comprise inorganic UV filters which have been aftertreated by conventional methods, as described, for example, in Cosmetics & Toiletries, February 1990, Vol. 105, pp. 53-64. One or more of the following aftertreatment components can be selected here: amino acids, beeswax, fatty acids, fatty acid alcohols, anionic surfactants, lecithin, phospholipids, sodium, potassium, zinc, iron or aluminium salts of fatty acids, polyethylenes, silicones, proteins (particularly collagen or elastin), alkanolamines, silicon dioxide, aluminium oxide, further metal oxides, phosphates, such as sodium hexametaphosphate, or glycerine.

Particulate UV filters which are preferably employed here are:
untreated titanium dioxides, such as, for example, the products Microtitanium Dioxide MT 500 B from Tayca; titanium dioxide P25 from Degussa,
aftertreated micronised titanium dioxides with aluminium oxide and silicon dioxide aftertreatment, such as, for example, the product “Microtitanium Dioxide MT 100 SA from Tayca; or the product “Tioveil Fin” from Uniqema,
aftertreated micronised titanium dioxides with aluminium oxide and/or aluminium stearate/laurate aftertreatment, such as, for example, Microtitanium Dioxide MT 100 T from Tayca, Eusolex T-2000 from Merck,
aftertreated micronised titanium dioxides with iron oxide and/or iron stearate aftertreatment, such as, for example, the product “Microtitanium Dioxide MT 100 F” from Tayca,
aftertreated micronised titanium dioxides with silicon dioxide, aluminium oxide and silicone aftertreatment, such as, for example, the product “Microtitanium Dioxide MT 100 SAS”, from Tayca,
aftertreated micronised titanium dioxides with sodium hexametaphosphates, such as, for example, the product “Microtitanium Dioxide MT 150 W” from Tayca.

The treated micronised titanium dioxides employed for the combination may also be aftertreated with:
octyltrimethoxysilanes; such as, for example, the product Tego Sun T 805 from Degussa,
silicon dioxide; such as, for example, the product Parsol T-X from DSM,
aluminium oxide and stearic acid; such as, for example, the product UV-Titan M160 from Sachtleben,
aluminium and glycerine; such as, for example, the product UV-Titan from Sachtleben,
aluminium and silicone oils, such as, for example, the product UV-Titan M262 from Sachtleben,
sodium hexametaphosphate and polyvinylpyrrolidone,
polydimethylsiloxanes, such as, for example, the product 70250 Cardre UF TiO2SI3″ from Cardre,
polydimethylhydrogenosiloxanes, such as, for example, the product Microtitanium Dioxide USP Grade Hydrophobic” from Color Techniques.

The combination with the following products may furthermore also be advantageous:
untreated zinc oxides, such as, for example, the product Z-Cote from BASF (Sunsmart), Nanox from Elementis
aftertreated zinc oxides, such as, for example, the following products:
“Zinc Oxide CS-5” from Toshibi (ZnO aftertreated with polymethylhydrogenosiloxanes)
Nanogard Zinc Oxide FN from Nanophase Technologies
“SPD-Z1” from Shin-Etsu (ZnO aftertreated with a silicone-grafted acrylic polymer, dispersed in cyclodimethylsiloxanes
“Escalol Z100” from ISP (aluminium oxide-aftertreated ZnO dispersed in an ethylhexyl methoxycinnamate/PVP-hexadecene/methicone copolymer mixture)
“Fuji ZNO-SMS-10” from Fuji Pigment (ZnO aftertreated with silicon dioxide and polymethylsilesquioxane);
untreated cerium oxide micropigment, for example with the name “Colloidal Cerium Oxide” from Rhone Poulenc
untreated and/or aftertreated iron oxides with the name Nanogar from Arnaud.

For example, it is also possible to employ mixtures of various metal oxides, such as, for example, titanium dioxide and cerium oxide, with and without aftertreatment, such as, for example, the product Sunveil A from Ikeda. In addition, it is also possible to use mixtures of aluminium oxide, silicon dioxide and silicone-aftertreated titanium dioxide. zinc oxide mixtures, such as, for example, the product UV-Titan M261 from Sachtleben, in combination with the UV protection agent according to the invention.

These inorganic UV filters are generally incorporated into the compositions in an amount of 0.1 percent by weight to 25 percent by weight, preferably 2% by weight—10% by weight.

By combination of one or more of the said compounds having a UV filter action, the protective action against harmful effects of the UV radiation can be optimised.

Preferred compositions may also comprise at least one further cosmetic active compound, for example selected from antioxidants, anti-aging active compounds, anti-cellulite active compounds, self-tanning substances, skin-lightening active compounds or vitamins.

Combinations according to the invention can furthermore be combined with all active compounds and assistants as listed systematically in WO2009/098139. In particular, these substances belong to the use categories mentioned therein “moisturizers and humectants”, “desquamating agents”, “agents for improving the barrier function”, “depigmenting agents”, “antioxidants”, “dermo-relaxing or dermo-decontracting agents”, “anti-glycation agents”, “agents for stimulating the synthesis of dermal and/or epidermal macromolecules and/or for preventing their degradation”, “agents for stimulating fibroblast or keratinocyte proliferation and/or keratinocyte differentiation”, “agents for promoting the maturation of the horny envelope”, “NO-synthase inhibitors”, “peripheral benzodiazepine receptor (PBR) antagonists”, “agents for increasing the activity of the sebaceous glands”, “agents for stimulating the energy metabolism of cells”, “tensioning agents”, “fat-restructuring agents”, “sliming agents”, “agents for promoting the cutaneous microcirculation”, “calmatives or anti-irritants”, “sebo-regulating or anti-seborrhoic agents”, “astringents”, “cicatrising agents”, “anti-inflammatory agents”, “antiacne agents”.

The protective action of compositions against oxidative stress or against the effect of free radicals can additionally be improved if the compositions described above comprise one or more antioxidants, the person skilled in the art being presented with absolutely no difficulties in selecting antioxidants which act suitably quickly or with a time delay.

There are many proven substances known from the specialist literature which can be used as antioxidants, for example amino acids (for example glycine, histidine, tyrosine, tryptophan) and derivatives thereof, imidazoles, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents, (for example α-hydroxyfatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate) and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiaretic acid, tri hydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Suitable antioxidants are also compounds of the formulae A or B

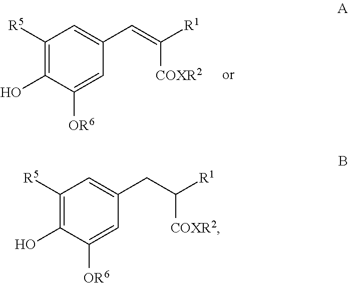

in which
R¹ can be selected from the group —C(O)CH₃, —CO₂R³, —C(O)NH₂ and —C(O)N(R⁴)₂,
X denotes O or NH,′
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁵ denotes H or linear or branched alkyl having 1 to 8 C atoms or linear or branched alkoxy having 1 to 8 C atoms and
R⁶ denotes linear or branched alkyl having 1 to 8 C atoms, preferably derivatives of 2-(4-hydroxy-3,5-dimethoxybenzylidene)malonic acid and/or 2-(4-hydroxy-3,5-dimethoxybenzyl)malonic acid, particularly preferably bis(2-ethylhexyl) 2-(4-hydroxy-3,5-dimethoxybenzylidene)malonate (for example Oxynex® ST Liquid) and/or bis(2-ethylhexyl) 2-(4-hydroxy-3,5-di-methoxybenzyl)malonate (for example RonaCare® AP). Furthermore, the combination with bisisopropyl 2-(4-hydroxy-3-methoxybenzylidene)malonate or bisisopropyl 2-(4-hydroxy-3-methoxybenzyl)malonate is preferred. An analogous situation applies to corresponding bisethyl esters.

The terms R¹ to R⁶ and X only apply here to the radicals of the formulae A and B and do not apply to the radicals of the formula I.

Mixtures of antioxidants are likewise suitable for use in the cosmetic compositions according to the invention. Known and commercial mixtures are, for example, mixtures comprising, as active ingredients, lecithin, L-(+)-ascorbyl palmitate and citric acid, natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed in such compositions with the compounds according to the invention in percent by weight ratios in the range from 1000:1 to 1:1000, preferably in percent by weight ratios of 100:1 to 1:100.

Of the phenols which can be used in accordance with the invention, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I.M.C.M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers and I. M. C. M. Rietjens (Free Radical Biology Medicine 2001, 31(7), 869-881, have investigated the pH dependence of the antioxidant action of hydroxyflavones. Quercetin exhibits the highest activity amongst the structures investigated over the entire pH range.

Suitable anti-aging active compounds, in particular for skin-care compositions, are preferably so-called compatible solutes. These are substances which are involved in the osmosis regulation of plants or microorganisms and can be isolated from these organisms. The generic term compatible solutes here also encompasses the osmolytes described in German patent application DE-A-10133202. Suitable osmolytes are, for example, the polyols, methylamine compounds and amino acids and respective precursors thereof. Osmolytes in the sense of German patent application DE-A-10133202 are taken to mean, in particular, substances from the group of the polyols, such as, for example, myo-inositol, mannitol or sorbitol, and/or one or more of the osmolytically active substances mentioned below: taurine, choline, betaine, phosphorylcholine, glycerophosphorylcholines, glutamine, glycine, α-alanine, glutamate, aspartate, proline and taurine. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

Compatible solutes which are preferably employed in accordance with the invention are substances selected from the group consisting of pyrimidine-carboxylic acids (such as ectoin and hydroxyectoin), proline, betaine, glutamine, cyclic diphosphoglycerate, N.-acetylornithine, trimethylamine N-oxide di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) or/and dimannosyl diinositol phosphate (DMIP) or an optical isomer, derivative, for example an acid, a salt or ester, of these compounds, or combinations thereof.

Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidine-carboxylic acid) and derivatives thereof.

Furthermore, the compositions according to the invention may comprise at least one self-tanning agent as further ingredient.

Advantageous self-tanning agents which can be employed are, inter alia: 1,3-dihydroxyacetone, glycerolaldehyde, hydroxymethylglyoxal, γ-dialdehyde, erythrulose, 6-aldo-D-fructose, ninhydrin, 5-hydroxy-1,4-naphthoquinone (juglone) or 2-hydroxy-1,4-naphthoquinone (lawsone). Very particular preference is given to 1,3-dihydroxyacetone, erythrulose or a combination thereof.

The compositions may also comprise one or more further skin-lightening active compounds or synonymously depigmentation active compounds. Skin-lightening active compounds can in principle be all active compounds known to the person skilled in the art. Examples of compounds having skin-lightening activity are hydroquinone, kojic acid, arbutin, aloesin or rucinol.

The compositions to be employed may comprise vitamins as further ingredients. Preference is given to vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine, (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. In the case of cosmetic application, vitamins are usually added with the flavonoid-containing premixes or compositions in ranges from 0.01 to 5.0% by weight, based on the total weight. Nutrition-physiological applications are oriented towards the respective recommended vitamin requirement.

The retinoids described are at the same time also effective anti-cellulite active compounds. A likewise known anti-cellulite active compound is caffeine.

The said constituents of the composition can be incorporated in the usual manner, with the aid of techniques which are well known to the person skilled in the art.

Suitable compositions are those for external application, for example can be sprayed onto the skin as cream or milk (O/W, W/O, O/W/O, W/O/W), as lotion or emulsion, in the form of oily-alcoholic, oily-aqueous or aqueous-alcoholic gels or solutions. They can be in the form of solid sticks or formulated as an aerosol. Administration forms such as capsules, dragees, powders, tablet solutions or solutions are suitable for internal use.

Examples which may be mentioned of application forms of the compositions to be employed are: solutions, suspensions, emulsions, PIT emulsions, pastes, ointments, gels, creams, lotions, powders, soaps, surfactant-containing cleansing preparations, oils, aerosols and sprays.

Preferred assistants originate from the group of preservatives, stabilisers, solubilisers, colorants, odour improvers.

Ointments, pastes, creams and gels may comprise the customary vehicles which are suitable for topical application, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary readily volatile, liquefied propellants, for example chlorofluorocarbons, propane/butane or dimethyl ether. Compressed air can also advantageously be used.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

A preferred solubiliser in general is 2-isopropyl-5-methylcyclohexanecarbonyl-D-alanine methyl ester.

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, powder make-up, emulsion make-up and wax make-up, and sunscreen, pre-sun and after-sun preparations.

The preferred composition forms also include, in particular, emulsions.

Emulsions are advantageous and comprise, for example, the said fats, oils, waxes and other fatty substances, as well as water and an emulsifier, as usually used for a composition of this type.

The lipid phase may advantageously be selected from the following group of substances:
mineral oils, mineral waxes
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group of esters of aromatic carboxylic acid and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The compositions according to the invention may preferably comprise assistants, such as, for example, cosmetic oils (for example Caprylic/Capric Triglycerides, C12-15 Alkyl Benzoate, isopropyl myristate, Arylalkyl Benzoate, such as, for example, phenethyl benzoate (X-Tend 226) or oil components of the Cosmacol brand, such as Dimyristyl Tartrate, Tri C14-C15 Alkyl Citrate, C12-C13 Alkyl Lactate, Tridecyl Salicylate, C12-C13 Alkyl Octanoate, C12-C13 Alkyl Malate, C12-C13 Alkyl Citrate, C12-C13 Alkyl Tartrate), or polar-protic assistants (for example propylene glycol, glycerine, isopropanol, ethanol) or so-called solubilisers (for example butylphthalimides, isopropylphthalimides, dimethylisosorbides).

The oil phase may furthermore advantageously be selected from the group branched and unbranched hydrocarbons and hydrocarbon waxes, silicone oils, dialkyl ethers, the group of saturated or unsaturated, branched or unbranched alcohols, and fatty acid triglycerides, specifically the triglyceryl esters of saturated and/or unsaturated, branched and/or unbranched alkanecarboxylic acids having a chain length of 8 to 24, in particular 12-18 C atoms. The fatty acid triglycerides may, for example, advantageously be selected from the group of synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as sole lipid component of the oil phase.

The aqueous phase of the compositions to be employed optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions are advantageous and comprise, for example, the said fats, oils, waxes and other fatty substances, as well as water and an emulsifier, as usually used for a formulation of this type.

In a preferred embodiment, the compositions to be employed comprise hydrophilic surfactants. The hydrophilic surfactants are preferably selected from the group of the alkylglucosides, acyl lactylates, betaines and coconut amphoacetates.

It is likewise advantageous to employ natural or synthetic raw materials and assistants or mixtures which are distinguished by an effective content of the active compounds used in accordance with the invention, for example Plantaren® 1200 (Henkel KGaA), Oramix® NS 10 (Seppic).

The cosmetic and dermatological compositions may exist in various forms. Thus, they may be, for example, a solution, a water-free composition, an emulsion or microemulsion of the water-in-oil (W/O) type or of the oil-in-water (O/W) type, a multiple emulsion, for example of the water-in-oil-in-water (W/O/W) type, a gel, a solid stick, an ointment or an aerosol. It is also advantageous to administer ectoins in encapsulated form, for example in collagen matrices and other conventional encapsulation materials, for example as cellulose encapsulations, in gelatine, wax matrices or liposomally encapsulated. In particular, wax matrices, as described in DE-A-43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions.

The co-emulsifiers selected are advantageously, for example, O/W emulsifiers, principally from the group of substances having HLB values of 11-16, very particularly advantageously having HLB values of 14.5-15.5, so long as the O/W emulsifiers have saturated radicals R and R′. If the O/W emulsifiers have unsaturated radicals R and/or R′, or if isoalkyl derivatives are present, the preferred HLB value of such emulsifiers may also be lower or higher.

It is advantageous to select the fatty alcohol ethoxylates from the group of the ethoxylated stearyl alchols, cetyl alcohols, cetylstearyl alcohols (cetearyl alcohols).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth1-4 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/cprinate, polyethylene glycol (20) glyceryl oleate, polyethylene glycol (20) glyceryl isostearate, polyethylene glycol (18) glyceryl oleate (cocoate).

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be employed as optional W/O emulsifiers, but ones which may nevertheless be advantageous in accordance with the invention:

fatty alcohols having 8 to 30 carbon atoms, monoglycerol esters of saturated and/or unsaturated, branched and/or unbranched alkanecarboxylic acids having a chain length of 8 to 24, in particular 12-18 C atoms, diglycerol esters of saturated and/or unsaturated, branched and/or unbranched alkanecarboxylic acids having a chain length of 8 to 24, in particular 12-18 C atoms, monoglycerol ethers of saturated and/or unsaturated, branched and/or unbranched alcohols having a chain length of 8 to 24, in particular 12-18 C atoms, diglycerol ethers of saturated and/or unsaturated, branched and/or unbranched alcohols having a chain length of 8 to 24, in particular 12-18 C atoms, propylene glycol esters of saturated and/or unsaturated, branched and/or unbranched alkanecarboxylic acids having a chain length of 8 to 24, in particular 12-18 C atoms, and sorbitan esters of saturated and/or unsaturated, branched and/or unbranched alkanecarboxylic acids having a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate or PEG-30 dipolyhydroxystearate.

The composition may comprise cosmetic adjuvants which are usually used in this type of composition, such as, for example, thickeners, softeners, moisturizers, surface-active agents, emulsifiers, preservatives, antifoams, perfumes, waxes, lanolin, propellants, dyes and/or pigments, and other ingredients usually used in cosmetics.

The dispersant or solubiliser used can be an oil, wax or other fatty substance, a lower monoalcohol or a lower polyol or mixtures thereof. Particularly preferred monoalcohols or polyols include ethanol, i-propanol, propylene glycol, glycerol and sorbitol.

A preferred embodiment of the invention is an emulsion which is in the form of a protective cream or milk and comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The composition may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a composition is formulated as an aerosol, use is generally made of the customary propellants, such as alkanes.

Even without further comments, it is assumed that a person skilled in the art will be able to utilise the above description in the broadest scope. The preferred embodiments and examples should therefore merely be regarded as descriptive disclosure which is absolutely not limiting in any way. The complete disclosure content of all applications and publications mentioned above and below is incorporated into this application by way of reference. The percent by weight ratios of the individual ingredients in the compositions of the examples expressly belong to the disclosure content of the description and can therefore be utilised as features.

Further important features and advantages of the invention arise from the sub-claims and from the examples.

It goes without saying that the features mentioned above and still to be explained below can be used not only in the respective combination indicated, but also in other combinations or in isolation without leaving the framework of the present invention.

Preferred embodiments of the invention are described in the examples and are explained in greater detail in the following description without restricting the scope of the present invention.
Ex. 1 shows an increasing UV absorption capacity of 3-(4-tert-butylphenyl)-3-hydroxy-1-(4-methoxyphenyl)propan-1-one through the addition of Uvinul® T150,
Ex. 2 shows a plurality of illustrative cosmetic compositions comprising at least one compound of the formula I and 3-(4-tert-butylphenyl)-3-hydroxy-1-(4-methoxyphenyl)propan-1-one.

### EXAMPLE 1

In a series of experiments, solution compositions shown below were each irradiated with UV light in a dose of 500 kJ/m², corresponding to 10 MED (minimal erythemal dose), using an Atlas Suntest CPS+ instrument. This irradiation converts 3-(4-tert-butylphenyl)-3-hydroxy-1-(4-methoxyphenyl)-propan-1-one (MeO-MBM) into butylmethoxydibenzoylmethane (avobenzone). The amount of avobenzone formed can be determined by evaluation of the characteristic UV absorption band at 358 nm. The cosmetic vehicles employed were Miglyol 812® (capry/capric acid triglyceride) from Sasol or Arlasolv DMI® (dimethylisosorbide) from Croda. Uvinul T150®, if used, was purchased from BASF. The 100% reference used in each case is the composition without Uvinul T150®.

| | Relative content of | Relative content of |
|---|---|---|
| | avobenzone before | avobenzone after |
| Solution composition | irradiation | irradiation |
| MeO-MBM (2 w %) in | 0% | 100% |
| Miglyol 812 ® | | |
| MeO-MBM (2 w %) and | 0% | 156% |
| Uvinul T150 (1 w %) in | | |
| Miglyol 812 ® | | |
| MeO-MBM (2 w %) in | 0% | 100% |
| Arlasolv DMI | | |
| MeO-MBM (2 w %) and | 0% | 178% |
| Uvinul T150 (1 w %) in | | |
| Arlasolv DMI | | |

It was found that combination according to the invention of MeO-MBM and Uvinul T150 in various oils unexpectedly accelerated the formation of avobenzone by photoconversion of MeO-MBM.

### Composition Examples

Composition examples shown below may furthermore comprise the following stabilisers (each 0.1-10% in individual component or in mixtures): Benzotriazolyl Dodecyl p-Cresol (Tinoguard TL), Butyloctyl salicylate, Diethylhexyl 2,6-Naphthalate, Diethylhexyl Syringylidene malonate, Polyester-8 (Polycrylene), bis-Ethylhexyl Hydroxydimethoxy Benzylmalonate.

### Example 1

### W/O Emulsion

| | 54-07-5-A | 54-07-5-B | 54-07-5-C | 54-07-5-D | 54-07-5-E |
|---|---|---|---|---|---|
| Cetyl PEG/PPG-10/1 | 3.00 | 3.00 | 3.00 | 3.00 | 3.00 |
| dimethicone | | | | | |
| (Abil EM 90) | | | | | |
| Polyglyceryl-4 | 1.50 | 1.50 | 1.50 | 1.50 | 1.50 |
| isostearate | | | | | |
| (Isolan GI 34) | | | | | |
| Butylphthalimide | 5.00 | 5.00 | 5.00 | 5.00 | 5.00 |
| isopropylphthalimide | | | | | |
| (Pelemol ® BIP) | | | | | |
| Dimethyl isosorbide | 5.00 | 5.00 | 5.00 | 5.00 | 5.00 |
| (Arlasolve DMI) | | | | | |
| 3-(4-tert-Butylphenyl)- | 1.00 | | | 1.00 | 2.00 |
| 3-hydroxy-1-(4- | | | | | |
| methoxyphenyl)- | | | | | |
| propan-1-one | | | | | |
| 1-(4-tert-Butyl- | 2.00 | 1.00 | 3.00 | | |
| phenyl)-3-hydroxy-3- | | | | | |
| (4-methoxyphenyl)- | | | | | |
| propan-1-one | | | | | |
| Uvinur ® A Plus | | 0.84 | 0.84 | 1.00 | |
| Formula XVI (Uvasorb | 2.0 | 2.0 | 1.5 | 4.0 | 5.0 |
| Heb) | | | | | |
| Formula X (Tinosorb | 2.0 | 2.0 | 3.0 | 1.5 | |
| S) | | | | | |
| Ascorbic acid | | | 0.37 | 1.00 | 3.00 |
| Mineral Oil | 8.00 | 8.00 | 8.00 | 8.00 | 8.00 |
| Ethylhexyl stearate | 5.00 | 5.00 | 5.00 | 5.00 | 5.00 |
| (Tegosoft ® OS) | | | | | |
| Cyclomethicone (and) | 5.00 | 5.00 | 5.00 | 5.00 | 5.00 |
| Aluminium/Magnesium | | | | | |
| Hydroxide Stearate | | | | | |
| (Gilugel SIL 5) | | | | | |
| Preservative | 1.00 | 1.00 | 1.00 | 1.00 | 1.00 |
| Water | To 100 | To 100 | To 100 | To 100 | To 100 |
| NaCl | 0.50 | 0.50 | 0.50 | 0.50 | 0.50 |
| EDTA | 0.10 | 0.10 | 0.10 | 0.10 | 0.10 |
| Citric acid q.S. | | | | | |

Preparation: Pelemol BIP, Arlasolv DMI and emulsifiers are initially introduced. 3-(4-tert-Butylphenyl)-3-hydroxy-1-(4-methoxyphenyl)propan-1-one, 1-(4-tert-butylphenyl)-3-hydroxy-3-(4-methoxy-phenyl)propan-1-one, Uvinul® A Plus and the trazines are dissolved therein. The remaining constituents of the oil phase are added and mixed homogeneously. The water phase, adjusted to pH=4-5, is emulsified in with stirring. The mixture is subsequently homogenised. The emulsions can be prepared under gentle conditions at room temperature.

### Example II

### Water-Resistant Sunscreen Spray

| A | | | |
|---|---|---|---|
| 3-(4-tert-Butylphenyl)-3- | 1.00 | 1.00 | 2.00 |
| hydroxy-1-(4-methoxy- | | | |
| phenyl)propan-1-one | | | |
| 1-(4-tert-Butylphenyl)-3- | | 2.00 | 1.50 |
| hydroxy-3-(4-methoxy- | | | |
| phenyl)propan-1-one | | | |
| Diethylhexyl | | 0.50 | |
| Syringylidenemalonate, | | | |
| Caprylic/Capric | | | |
| Triglyceride | | | |
| (Oxynex ® ST Liquid) | | | |
| RonaCare ® AP | | 2.00 | |
| Formula X (Tinsorb S) | 1.5 | 1.0 | 1.5 |
| Formula XVI (Uvasorb | 1.5 | 3.0 | 5.0 |
| Heb) | | | |
| Formula XVII (Uvinul | 1.5 | 0.5 | |
| T150) | | | |
| Ascorbyl Palmitate | | | 1.00 |
| Cyprylic/capric Triglyceride | 7.00 | 7.00 | 7.00 |
| (Miglyol 812 N) | | | |
| Butylphthalimide | 10.00 | 10.00 | 10.00 |
| isopropylphthalimide | | | |
| (Pelemol ® BIP) | | | |
| C12-15 alkyl benzoate | 10.00 | 10.00 | 10.00 |
| (Tegosoft ® TN) | | | |
| Phenethyl benzoate | 5.00 | 5.00 | 5.00 |
| (X-Tend 226) | | | |
| RonaCare ® tocopherol | 1.00 | 1.00 | 1.00 |
| acetate | | | |
| B | | | |
| Cyclopentasiloxane | 43.80 | 41.30 | 41.80 |
| (Dow Corning 245) | | | |
| Phenyltrimethicone | 2.00 | 2.00 | 2.00 |
| (Dow Corning 556) | | | |
| Cyclopentasiloxane, | 20.00 | 20.00 | 20.00 |
| dimethiconol | | | |
| Dow Corning 1501 Fluid | | | |
| Perfume oil (q.s.) | 0.20 | 0.20 | 0.20 |

Preparation: the components of phase A are combined at room temperature and stirred. Phase B is subsequently mixed and added to phase B with stirring, and the mixture is stirred.

### Example III

### Pump Hairspray

| | | | |
|---|---|---|---|
| A | | | |
| 3-(4-tert-Butylphenyl)-3-hydroxy- | 1.00 | 2.00 | 4.00 |
| 1-(4-methoxyphenyl)propan-1- | | | |
| one | | | |
| Formula XII | 1.00 | 1.00 | 1.00 |
| Formula X | | 1.00 | |
| 1-(4-tert-Butylphenyl)-3-hydroxy- | 1.00 | | 1.50 |
| 3-(4-methoxyphenyl)propan-1- | | | |
| one | | | |
| Ethanol 96% extra pure | To 100 | To 100 | To 100 |
| PVP/VA copolymer | 6.00 | 6.00 | 6.00 |
| PVP/VA W 735 | | | |
| B | | | |
| Diethylhexyl Syringylidenemalonate, | 0.06 | 0.25 | 0.50 |
| Caprylic/Capric Triglyceride | | | |
| (Oxynex ® ST Liquid) | | | |
| PEG-75 Lanolin | 0.20 | 0.20 | 0.20 |
| BHT | | | |
| (Solan E - Low Dioxane) | | | |
| Perfume | 0.10 | 0.10 | 0.10 |
| (Frag 280853 Green Activating) | | | |
| C | | | |
| Water, demineralised | 13.00 | 13.00 | 13.00 |
| Titriplex III | 0.10 | 0.10 | 0.10 |
| PEG-12 dimethicone | 0.50 | 0.50 | 0.50 |
| Dow Corning 193 Fluid | | | |
| 0.1% D&C Red No 33 (CI 17200) | 0.20 | 0.20 | 0.20 |
| in water | | | |
| PEG-40 Hydrogenated Castor Oil | 1.00 | 1.00 | 1.00 |
| (Cremophor RH 410) | | | |

Preparation: pre-dissolve phase A. Add phase B to phase A with stirring. Pre-mix phase C and add to the remainder, stir until a homogeneous mixture has formed.

### Example IV

### W/O Emulsions

| Emulsion | A | B | C | D | E | F |
|---|---|---|---|---|---|---|
| Polyglyceryl 2- | 3 | 5 | 3 | | | |
| dipolyhydroxystearate | | | | | | |
| PEG-30 dipolyhydroxystearate | | | 2 | 3 | 4 | 5 |
| Sodium starch octenylsuccinate | 0.5 | 0.4 | | 0.3 | | 1 |
| Glycine | 0.3 | 0.3 | 0.5 | 0.4 | | |
| Alcohol | | 5 | 2 | 5 | 4 | |
| Magnesium sulfate | 0.2 | 0.3 | 0.3 | 0.4 | 0.5 | 0.2 |
| C₁₂₋₁₅ alkyl Benzoate | 5 | 3 | | | 5 | |
| C₁₂₋₁₃ alkyl Tartrate | | 2 | | | | |
| Butylene glycol | 5 | | | | 3 | 3 |
| dicaprylate/dicaprate | | | | | | |
| Dicaprylyl Ether | | | | | 2 | |
| Mineral oil | | 4 | | 6 | | 8 |
| Octyldodecanol | 2 | | | | | |
| Dicapryl caprate | | 2 | | | 2 | 2 |
| Cyclomethicone | 5 | | 5 | 10 | | |
| Dimethicone | | | | 5 | | |
| Isohexadecane | | 1 | | | | |
| Butylene glycol | 5 | 8 | | | | 3 |
| Propylene glycol | | | 1 | | 5 | 3 |
| Glycerine | 3 | 5 | 7 | 10 | 3 | 3 |
| C18-38 acid triglycerides | 0.5 | | 1 | | 1 | |
| Titanium dioxide | 5 | 6 | 4 | | | 4 |
| Zinc oxide | 5 | | | | | |
| Formula X (Tinsosorb S) | | 3 | 3 | 2 | | |
| Formula XVII (Uvinul T150) | | 4.5 | 3 | | 3 | |
| 3-(4-tert-Butylphenyl)-3- | 2.0 | 0.5 | 1.0 | 1.0 | 3.0 | |
| hydroxy-1-(4-methoxy- | | | | | | |
| phenyl)propan-1-one | | | | | | |
| 1-(4-tert-Butylphenyl)-3- | | 1.5 | 1.0 | | | 3.0 |
| hydroxy-3-(4-methoxy- | | | | | | |
| phenyl)propan-1-one | | | | | | |
| Formula XVI (Uvasorb Heb) | | | 1.5 | 4 | | |
| Butyl methoxydibenzoyl- | 2 | 3 | 4 | | 1 | 3 |
| methane | | | | | | |
| Uvinul ® A Plus | | | | 4 | 2 | |
| Ethylhexyl methoxycinnamate | | | | | 7 | 5 |
| Benzotriazole coupled to | 4 | | 6 | | | |
| gelatine | | | | | | |
| Taurine | 0.1 | | | 0.5 | 0.2 | |
| Vitamin E acetate | 0.2 | 02 | | 0.3 | 0.1 | 0.5 |
| Na₂H₂EDTA | 0.1 | 0.1 | 0.2 | 0.2 | 0.2 | 0.5 |
| C8-C16 alkylpolyglycosides | 1 | | | | | |
| Perfume, preservative | q.s. | q.s | q.s | q.s | qs. | qs. |
| Dyes, etc. | q.s. | q.s. | q.s. | q.s | q.s. | q.s. |
| Sodium hydroxide | q.s. | q.s. | q.s. | q.s | q.s. | q.s. |
| Water | to 100.0 | to 100.0 | to 100.0 | to 100.0 | to 100.0 | to 100.0 |

### Example V

### Hair Care Formulation

Content in g of component per 100 g of formulation

| Component | A | B | C | D | E | F |
|---|---|---|---|---|---|---|
| Disodium EDTA | 0.100 | 0.100 | 0.100 | 0.100 | 0.100 | 0.100 |
| Oxynex ® ST | 2.000 | 2.000 | 2.000 | 2.000 | 2.000 | 2.000 |
| 3-(4-tert-Butylphenyl)-3-hydroxy- | 1.5 | 0.25 | 0.50 | 1.50 | 2.00 | 4.00 |
| 1-(4-methoxyphenyl)propan-1- | | | | | | |
| one | | | | | | |
| 1-(4-tert-Butylphenyl)-3- | | 1.75 | 2.5 | | | |
| hydroxy-3-(4-methoxyphenyl)- | | | | | | |
| propan-1-one | | | | | | |
| Formula XVII (Uvinul T150) | 0.50 | 0.50 | 0.50 | 0.50 | 0.50 | 0.50 |
| Formula XII | 0.50 | 0.50 | 0.50 | 0.50 | 0.50 | 0.50 |
| Hexamidine diisethionate | 0.100 | 0 | 0 | 0 | 0 | 0 |
| Tetrahydrocurcumin | 0 | 0.500 | 0 | 0 | 0 | 0 |
| Glycyrrhetinic acid | 0 | 0 | 0.300 | 0 | 0 | 0 |
| Thiotaine ®¹ | 0 | 0 | 0 | 5.000 | 0 | 0 |
| N-Undecylenoyl-L-phenylalanine | 0 | 0 | 0 | 0 | 1.000 | 0 |
| N-Acetyl glucosamine | 0 | 0 | 0 | 0 | 0 | 2.000 |
| Niacinamide | 5.000 | 5.000 | 5.000 | 5.000 | 5.000 | 5.000 |
| Citric acid | 0.015 | 0 | 0 | 0 | 0 | 0 |
| Isohexadecane | 3.000 | 3.000 | 3.000 | 3.000 | 3.000 | 3.000 |
| Isopropyl isostearate | 1.330 | 1.330 | 1.330 | 1.330 | 1.330 | 1.330 |
| Isopropyl N-laurosylsarcosinate | 0 | 0 | 5.000 | 0 | 0 | 0 |
| Sucrose polycottonseedate | 0.670 | 0.670 | 0.670 | 0.670 | 0.670 | 0.670 |
| Polymethylsilsesquioxane | 0.250 | 0.250 | 0.250 | 0.250 | 0.250 | 0.250 |
| Cetearyl glucoside + cetearyl | 0.200 | 0.200 | 0.200 | 0.200 | 0.200 | 0.200 |
| alcohol | | | | | | |
| Behenyl alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerine | 7.000 | 7.000 | 7.000 | 7.000 | 7.000 | 7.000 |
| Titanium dioxide | 0.604 | 0.604 | 0.604 | 0.604 | 0.604 | 0.604 |
| Polyacrylamide + C13-14 | 3.000 | 2.000 | 2.000 | 2.000 | 2.000 | 2.000 |
| isoparaffin + laureth-7 | | | | | | |
| Panthenol | 1.000 | 1.000 | 1.000 | 1.000 | 1.000 | 1.000 |
| Benzyl alcohol | 0.400 | 0.400 | 0.400 | 0.400 | 0.400 | 0.400 |
| Dimethicone + dimethiconol | 2.000 | 2.000 | 2.000 | 2.000 | 2.000 | 2.000 |
| Water (to 100 g) | to 100 | to 100 | to 100 | to 100 | to 100 | to 100 |
| TOTAL | 100 | 100 | 100 | 100 | 100 | 100 |

### Example VI

### Hair Care Formulation

Content in g of component per 100 g of formulation

| Component | G | H | I |
|---|---|---|---|
| Disodium EDTA | 0.100 | 0.100 | 0.100 |
| Oxynex ® ST | 2.000 | 2.000 | 2.000 |
| Formula X (Tinsorb S) | 1.00 | 1.00 | 1.00 |
| Formula XVII (Uvinul T150) | 1.00 | 1.00 | 1.00 |
| 3-(4-tert-Butylphenyl)-3-hydroxy-1-(4- | | 3.50 | 1.50 |
| methoxyphenyl)propan-1-one | | | |
| 1-(4-tert-Butylphenyl)-3-hydroxy-3-(4- | 2.00 | | |
| methoxyphenyl)propan-1-one | | | |
| Cetyl pyridinium chloride | 0.200 | 0 | 0 |
| Pitera ® | 0 | 10 | 0 |
| Ascorbyl glycoside | 0 | 0 | 2.000 |
| Niacinamide | 5.000 | 5.000 | 5.000 |
| Polyquaternium 37 | 0 | 0 | 0 |
| Isohexadecane | 3.000 | 3.000 | 3.000 |
| Isopropyl isostearate | 1.330 | 1.330 | 1.330 |
| Sucrose polycottonseedate | 0.670 | 0.670 | 0.670 |
| Polymethylsilsesquioxane | 0.250 | 0.250 | 0.250 |
| Cetearyl glucoside + cetearyl alcohol | 0.200 | 0.200 | 0.200 |
| Behenyl alcohol | 0.400 | 0.400 | 0.400 |
| Ethylparaben | 0.200 | 0.200 | 0.200 |
| Propylparaben | 0.100 | 0.100 | 0.100 |
| Cetyl alcohol | 0.320 | 0.320 | 0.320 |
| Stearyl alcohol | 0.480 | 0.480 | 0.480 |
| Tocopheryl acetate | 0.500 | 0.500 | 0.500 |
| PEG-100 stearate | 0.100 | 0.100 | 0.100 |
| Glycerine | 7.000 | 7.000 | 7.000 |
| Titanium dioxide | 0.604 | 0.604 | 0.604 |
| Polyacrylamide + C13-14 isoparaffin + | 2.000 | 2.000 | 2.000 |
| laureth-7 | | | |
| Panthenol | 1.000 | 1.000 | 1.000 |
| Benzyl alcohol | 0.400 | 0.400 | 0.400 |
| Dimethicone + dimethiconol | 2.000 | 2.000 | 2.000 |
| Water (to 100 g) | to 100 | to 100 | to 100 |
| TOTAL | 100 | 100 | 100 |

### Example VII

### O/W Emulsions

| Emulsion | A | B | C | D | E | F |
|---|---|---|---|---|---|---|
| Glyceryl stearate citrate | 2.5 | 2 | 3 | | | |
| Sorbitan stearate | 0.5 | | | 2 | 1.5 | 2 |
| Polyglyceryl-3 methylglycose | | | | 2.5 | 3 | 3 |
| distearate | | | | | | |
| Polyglyceryl-2 | | 0.8 | | | | 0.5 |
| dipolyhydroxystearate | | | | | | |
| Cetearyl alcohol | | | | 1 | | |
| Stearyl alcohol | 2 | | | | | 2 |
| Cetyl alcohol | | 1 | | | 3 | |
| Acrylates/C₁₀₋₃₀ Alkyl | | 0.2 | | | 0.1 | |
| Acrylate Crosspolymer | | | | | | |
| Carbomer | | 0.2 | 0.3 | 0.2 | | |
| Xanthan Gum | 0.4 | | 0.2 | 0.2 | 0.3 | 0.4 |
| C₁₂₋₁₅ alkyl benzoate | 5 | 3 | | | 5 | |
| C₁₂₋₁₃ alkyl tartrate | | 2 | | | | |
| Butylene glycol | 5 | | | | 3 | 3 |
| dicaprylate/dicaprate | | | | | | |
| Dicaprylyl Ether | | | | | 2 | |
| Octyldodecanol | 2 | | | | | |
| Dicapryl caprate | | 2 | | | 2 | 2 |
| Cyclomethicone | 5 | | 5 | 10 | | |
| Dimethicone | | | | 5 | | |
| Isohexadecane | | 1 | | | | |
| Butylene glycol | 5 | 8 | | | | 3 |
| Propylene gycol | | | 1 | | 5 | 3 |
| Glycerine | 3 | 5 | 7 | 10 | 3 | 3 |
| C18-C38 acid triglycerides | 0.5 | | 1 | | 1 | |
| Titanium dioxide | 5 | | | 2 | | |
| 2,2′-Methylenebis(6-(2H- | 2.5 | | | | | |
| benzotriazol-2-yl)-(1,1,3,3- | | | | | | |
| tetramethylbutyl)phenol) | | | | | | |
| Formula XII | | 2 | | | | |
| Formula XVI (Uvasorb Heb) | 4 | | 3 | | 3 | 2 |
| Benzotriazole coupled to | | 5 | | 10 | | 3 |
| gelatine | | | | | | |
| C8-C16 alkylpolyglycosides | 1 | 0.6 | | | | |
| Formula XV | | | 2 | | | |
| Uvinul ® A Plus | 2 | | | | | 1 |
| Homosalate | | 5 | | 1 | | |
| Phenylbenzimidazolesulfonic | | | 2 | | | 1 |
| acid | | | | | | |
| Benzophenone-3 | 2 | | | | 2 | |
| Octyl salicylate | 5 | 5 | | 2 | | |
| Octocrylene | 2 | | | | 3 | 1 |
| 3-(4-tert-Butylphenyl)-3- | | 2.0 | 3.0 | 1.0 | 2.0 | 3.0 |
| hydroxy-1-(4-methoxy- | | | | | | |
| phenyl)propan-1-one | | | | | | |
| 1-(4-tert-Butylphenyl)-3- | 3.0 | | | 2.0 | | |
| hydroxy-3-(4-methoxy- | | | | | | |
| phenyl)propan-1-one | | | | | | |
| Formula X (Tinsosorb S) | 1.2 | 3 | 2 | 1 | | |
| Parsole ® SLX | | | 3 | | | |
| Dihydroxyacetate | | | | | 4 | |
| Taurine | 0.1 | | | 0.5 | 0.2 | |
| 8-Hexadecene-1,16- | | 0.2 | | | | |
| dicarboxylic acid | | | | | | |
| Vitamin E acetate | 0.2 | 0.2 | | 0.3 | 0.1 | 0.5 |
| Na₂H₂EDTA | 0.1 | 0.1 | 0.2 | 0.2 | 0.2 | 0.5 |
| Perfume, preservative | q.s. | q.s. | q.s. | q.s. | q.s. | q.s. |
| Dyes, etc. | q.s. | q.s. | q.s. | q.s. | q.s. | q.s. |
| Sodium hydroxide | q.s. | q.s. | q.s. | q.s. | q.s. | q.s. |
| Water | to 100.0 | to 100.0 | to 100.0 | to 100.0 | to 100.0 | to 100.0 |

### Example VIII

### O/W Emulsions

| Emulsion | G | H | I | K | L | M |
|---|---|---|---|---|---|---|
| Ceteareth-20 | 1 | 1.5 | 1 | | | |
| Sorbitan stearate | | | 0.5 | | 0.5 | |
| Glyceryl Stearat SE | | | | 1 | 1 | 1.5 |
| Emulgade F ® | | | | 2.5 | 2.5 | 3 |
| Cetearyl alcohol | | | | 1 | | |
| Stearyl alcohol | | | | | 1.5 | |
| Cetyl alcohol | | | 0.5 | | | 2 |
| Acrylates/C₁₀₋₃₀ Alkyl | 0.2 | 0.4 | 0.3 | 0.1 | | |
| Acrylate Crosspolymer | | | | | | |
| Carbomer | | | | | 0.3 | |
| Xanthan Gum | | | | 0.4 | | 0.4 |
| C₁₂₋₁₅ alkyl benzoate | 5 | 3 | | | 5 | |
| 2-Phenyl benzoate | | 2 | | | | |
| Butylene glycol | 5 | | | | 3 | 2 |
| dicaprylate/dicaprate | | | | | | |
| Dicaprylyl Ether | | | | | 2 | |
| Diethylhexyl naphthalate | 2 | | | | | |
| Dicapryl caprate | | 2 | | | 2 | 2 |
| Cyclomethicone | 5 | | 5 | 10 | | |
| Isohexadecane | | | | 5 | | |
| Mineral oil | | 1 | | | | |
| Propylene glycol | | | 4 | | | |
| Glycerine | 5 | 7 | 3 | 5 | 6 | 8 |
| C18-38 acid triglycerides | 0.5 | | 1 | | 1 | |
| Titanium dioxide | 5 | | 3 | 2 | | |
| NeoHeliopan ® AP | | 2 | | | 1 | 1 |
| Phenylbenzimidazolesulfonic | 1 | | | 1 | 2 | 1 |
| acid | | | | | | |
| Ethylhexyl methoxy- | 5 | | 4 | 4 | | |
| cinnamate | | | | | | |
| Formula XVII (Uvinul T150) | | 2 | | 1 | | |
| Formula XVI (Uvasorb Heb) | 1 | | | | 3 | |
| Butyl | 2.5 | | 2 | 2 | | 1 |
| Methoxydibenzoylmethane | | | | | | |
| Formula X (Tinsosorb S) | 2 | 0.5 | 1.5 | | | 2.0 |
| 4-Methylbenzylidene | 3 | | | | | |
| Camphor | | | | | | |
| Parsol ® SLX | | | | | 2 | |
| 3-(4-tert-Butylphenyl)-3- | 1.0 | 2.0 | 4.0 | | 1.5 | 3.0 |
| hydroxy-1-(4-methoxy- | | | | | | |
| phenyl)propan-1-one | | | | | | |
| 1-(4-tert-Butylphenyl)-3- | 1.0 | | | 5.0 | | |
| hydroxy-3-(4-methoxy- | | | | | | |
| phenyl)propan-1-one | | | | | | |
| Creatinine | 0.1 | 0.01 | 0.05 | | | |
| Creatine | 0.5 | 0.2 | 0.1 | | | |
| Liquorice extract/ | | | | 0.5 | | |
| licochalcone | | | | | | |
| Vitamin E acetate | 0.2 | | | 0.5 | 0.5 | 0.5 |
| Tapioca starch | | 3 | | | 2 | |
| Na₂H₂EDTA | 0.1 | | 0.2 | | | 0.5 |
| Perfume, preservative | q.s. | q.s. | q.s. | q.s. | q.s. | q.s. |
| Dyes, etc. | q.s. | q.s. | q.s. | q.s. | q.s. | q.s. |
| Sodium hydroxide | q.s. | q.s. | q.s. | q.s. | q.s. | q.s. |
| Water | to 100.0 | to 100.0 | to 100.0 | to 100.0 | to 100.0 | to 100.0 |

### Example IX

### O/W Emulsions

| Emulsion | N | O | P | Q | R | S |
|---|---|---|---|---|---|---|
| Glyceryl stearate SE | | 2 | | 2 | | |
| Glyceryl stearate | 2 | | 2 | | | |
| PEG-40 stearate | | | 2 | | 1 | |
| PEG-10 stearate | | | | 2.5 | 1 | |
| Ceteareth-20 | | | | | | 2.6 |
| Sodium Cetyl Phosphate | | | | | 2 | |
| Glyceryl Stearate, | | | | | | 5.4 |
| Ceteareth-12, Ceteareth-20, | | | | | | |
| Cetearyl Alcohol, Cetyl | | | | | | |
| Palmitate | | | | | | |
| Stearic acid | 3 | 2 | | | 2 | |
| Stearyl alcohol | | 2 | 2 | | | |
| Stearyl alcohol | 0.5 | | 2 | | | |
| Cetyl alcohol | 3 | | | 2 | | |
| Acrylates/C10-30 Alkyl | | | 0.2 | | 0.4 | |
| Acrylate Crosspolymer | | | | | | |
| Carbomer | | 0.3 | | 0.3 | 0.3 | |
| Xanthan Gum | | 0.3 | 0.4 | | | |
| C₁₂₋₁₅ alkyl benzoate | 5 | | | | 5 | 3 |
| 2-Phenyl benzoate | 5 | | | | | |
| Butylene glycol | | 5 | | 4 | | 3 |
| dicaprylate/dicaprate | | | | | | |
| Dicaprylyl Ether | | 2 | | | 3 | |
| Diethylhexyl naphthalate | 3 | | | | | |
| Cyclomethicone | 2 | | 10 | 2 | | |
| Isohexadecane | | | | 2 | 3 | |
| Mineral oil | | | | | 3 | |
| Propanediol | | 3 | | 5 | | |
| Glycerine | 3 | 5 | 10 | 7 | 4 | 5 |
| Titanium dioxide | 2 | 4 | | | | |
| Zinc oxide | | | | | 2 | |
| Drometrizole Trisiloxane | | | | | 3 | |
| Ethylhexyl methoxy- | | 6 | 5 | | | |
| cinnamate | | | | | | |
| Phenylbenzimidazolesulfonic | | 0.5 | 2 | | 1 | |
| acid | | | | | | |
| Homosalate | 5 | | | 7 | | |
| Butyl | | 3 | | | | |
| Methoxydibenzoylmethane | | | | | | |
| Formula X (Tinsorb S) | | 2 | 3 | | | |
| Formula XVI (Uvasorb Heb) | 1.5 | 1.5 | 1.5 | 1.5 | 1.5 | 1.5 |
| Octyl salicylate | | | | 5 | | |
| Octocrylene | 1.0 | | | | 3 | |
| 3-(4-tert-Butylphenyl)-3- | | 1.5 | 0.5 | 2.5 | 1.0 | 5.0 |
| hydroxy-1-(4-methoxy- | | | | | | |
| phenyl)propan-1-one | | | | | | |
| 1-(4-tert-Butylphenyl)-3- | 3.0 | | 2.5 | | | |
| hydroxy-3-(4-methoxy- | | | | | | |
| phenyl)propan-1-one | | | | | | |
| Parsol ® SLX | 4 | | | | | 5 |
| PVP hexadecene copolymer | 0.5 | | 1 | | 0.8 | |
| Coenzyme Q 10 | 0.2 | 0.02 | | 0.3 | | |
| Vitamin E acetate | 0.2 | | 0.3 | | 0.8 | 0.5 |
| Na₂H₂EDTA | 0.1 | | | | | 0.5 |
| Perfume, preservative | q.s. | q.s. | q.s. | q.s. | q.s. | q.s. |
| Dyes, etc. | q.s. | q.s. | q.s. | q.s. | q.s. | q.s. |
| Sodium hydroxide | q.s. | q.s. | q.s. | q.s. | q.s. | q.s. |
| Water | to 100.0 | to 100.0 | to 100.0 | to 100.0 | to 100.0 | to 100.0 |

### Example X

### Hydrodisperions (Lotions and Sprays)

| | A | B | C | D | E | F |
|---|---|---|---|---|---|---|
| Glyceryl stearate citrate | | 0.40 | | | | |
| Cetyl alcohol | | | | | 2.00 | |
| Sodium Carbomer | | | | | 0.30 | |
| Acrylates/C₁₀₋₃₀ Alkyl | 0.30 | | 0.30 | 0.40 | 0.10 | 0.10 |
| Acrylate Crosspolymer | | | | | | |
| Cetsareth-20 | | | 1.00 | | | |
| Xanthan gum | | | | 0.15 | | 0.50 |
| Dimethicone/Vinyl | | | | 5.00 | | 3.00 |
| Dimethicone Crosspolymer | | | | | | |
| Formula XV (Uvasorb K2A) | | | | | 3.50 | |
| Uvinul ® A Plus | 0.25 | | | 0.50 | 2.00 | 1.50 |
| Butyl | 1.20 | | 3.50 | | | |
| Methoxydibenzoylmethane | | | | | | |
| Formula X (Tinsosorb S) | 2.00 | 2.00 | | 0.25 | | |
| Terephthalidenedicamphor- | | | | | | 0.50 |
| sulfonic acid | | | | | | |
| Disodium Phenyl | | | | | | 1.00 |
| Dibenzimidazole | | | | | | |
| Tetrasulfonate | | | | | | |
| Phenylbenzimidazolesulfonic | | | 2.00 | | | |
| acid | | | | | | |
| Ethylhexyl methoxy- | 5.00 | | 7.00 | | 5.00 | 8.00 |
| cinnamate | | | | | | |
| Formula XVI (Uvasorb Heb) | | | 2.00 | 2.00 | | |
| Formula XVII (Uvinul T150) | 4.00 | 3.00 | 0.5 | | 4.00 | |
| Octocrylene | | | | 10.00 | | 2.50 |
| 3-(4-tert-Butylphenyl)-3- | 0.25 | 1.5 | | 2.5 | 1.0 | 5.0 |
| hydroxy-1-(4-methoxy- | | | | | | |
| phenyl)propan-1-one | | | | | | |
| 1-(4-tert-Butylphenyl)-3- | 1.75 | | 2.0 | 2.5 | | |
| hydroxy-3-(4-methoxy- | | | | | | |
| phenyl)propan-1-one | | | | | | |
| C₁₂₋₁₅ alkyl benzoate | 2.00 | | 2.50 | | | |
| Phenethyl benzoate | 4.00 | | | 7.50 | | 5.00 |
| C₁₈₋₃₆ triglyceride fatty acid | | | 1.00 | | | |
| Butylene glycol | | | | | 6.00 | |
| dicaprylate/dicaprate | | | | | | |
| Dicaprylyl carbonate | | 3.00 | | | | |
| Dicaprylyl ether | | 2.00 | | | | |
| Cyclomethicone | | | | 1.50 | | |
| Lanolin | | | | | 0.35 | |
| PVP hexadecene copolymer | 0.50 | | 0.50 | | 0.50 | 1.00 |
| Ethylhexyloxyglycerine | | 0.75 | | 1.00 | | 0.50 |
| Glycerine | 10.00 | 5.00 | 5.00 | | 5.00 | 15.00 |
| Butylene glycol | | 7.00 | | | | |
| Glycine soya | | | | 1.00 | | |
| Vitamin E acetate | 0.50 | 0.25 | 0 50 | 0.25 | 0.75 | 1.00 |
| α-Glycosylrutin | | | | | 0.25 | |
| Trisodium EDTA | | 1.00 | 1.00 | 0.10 | 0.20 | |
| Idopropinyl butylcarbamate | 0.20 | 0.10 | | | | 0.15 |
| Methylparaben | 0.50 | | 0.20 | | 0.15 | |
| Phenoxyethanol | 0.50 | 0.40 | 0.40 | | 1.00 | 0.60 |
| Ethanol | 3.00 | 10.00 | 4.00 | 3.50 | | 1.00 |
| Perfume, dyes | q.s. | q.s. | q.s. | qs. | q.s. | q.s. |
| Water | to 100 | to 100 | to 100 | to 100 | to 100 | to 100 |
| Neutraliser (sodium | qs | qs | qs | qs | qs | qs |
| hydroxide, potassium | | | | | | |
| hydroxide) | | | | | | |

### Example XI

### Aqueous and Aqueous/Alcoholic Formulations

| | A | E | C | D | E | F |
|---|---|---|---|---|---|---|
| Ethanol | 50 | 5 | 2 | 40 | 15 | |
| Hydroxyethylcellulose | 0.5 | | | | | |
| Acrylates/C10-30 Alkyl Acrylate | | | | 0.3 | 0.6 | |
| Crosspolymer | | | | | | |
| Cocoatnidopropylbetain | | | 0.3 | | | |
| Formula XV (Uvasorb K2A) | | | | | 2 | |
| Uvinul ® APlus | 5 | | | | | |
| Butyl | 0.5 | | | 3 | | |
| Methoxydibenzoylmethane | | | | | | |
| Disodium phenyl- | | 2 | 1 | | | |
| dibenzimidazoletetrasulfonate | | | | | | |
| Phenylbenzimidazolesulfonic | | 5 | 3 | | 2 | 4 |
| acid | | | | | | |
| Ethylhexyl Methoxycinnamat | 10 | | | | 3 | |
| Formula XVI (Uvasorb Heb) | | 1 | | 3 | | |
| Formula XVII (Uvinul T150) | | 1 | | | 2 | |
| Octocrylene | | | | 5 | | |
| 3-(4-tert-Butylphenyl)-3- | 2.5 | | 1.5 | 3.0 | 3.5 | 4.0 |
| hydroxy-1-(4-methoxyphenyl)- | | | | | | |
| propan-1-one | | | | | | |
| 1-(4-tert-Butylphenyl)-3- | 2.5 | 3.0 | | | | 1.0 |
| hydroxy-3-(4-methoxy- | | | | | | |
| phenyl)propan-1-one | | | | | | |
| C₁₂₋₁₅ alkyl benzoate | | | | 3 | | |
| C18-36 triglyceride fatty acid | | | | 1 | | |
| Butylene glycol | 2 | | | | | |
| dicaprylate/dicaprate | | | | | | |
| C12-13 alkyl tartrate | | | | | 5 | |
| Cyclomethicone | 4 | | | 2 | | |
| Insect repellent ® 3535 | | | | 5 | | |
| Dimethicone | | | | | 3 | |
| PVP hexadecene copolymer | | 0.5 | | 1 | | 0.5 |
| Ethylhexyloxyglycerine | | 0.5 | | | | |
| Glycerine | 5 | 7 | 3 | 8 | | S |
| Butylene glycol | | | 5 | | 5 | |
| Metylpropanediol | | | | 4 | | |
| Vitamin E acetate | | 0.3 | 0.2 | 0.5 | | |
| Panthenol | 0.5 | | 0.2 | | | 0.3 |
| Creatinine | | | 0.01 | | 0.02 | |
| Creatine | | | 0.1 | | 0.2 | |
| PEG-40 hydrogenated castor | | 0.5 | 0.3 | | | 0.5 |
| oil | | | | | | |
| Trisodium EDTA | 0.3 | 0.2 | 0.2 | 0.2 | 0.2 | 0.5 |
| Preservative | q.s. | q.s. | q.s. | q.s. | q.s. | q.s. |
| Sodium hydroxide | q.s. | q.s. | q.s. | q.s. | q.s. | q.s. |
| Perfum, dyes | q.s. | q.s. | q.s. | q.s. | q.s. | q.s. |
| Water | to 100 | to 100 | to 100 | to 100 | to 100 | to 100 |

### Example XII

### Cosmetic Foams

| Emulsion | A | B | C |
|---|---|---|---|
| Stearic acid | 2 | 2 | |
| Palmitic acid | | | 1.5 |
| Cetyl alcohol | 2.5 | 2 | |
| Stearyl alcohol | | | 3 |
| PEG-100 stearate | | | 3.5 |
| PEG-40 stearate | | 2 | |
| PEG-20 stearate | 3 | | |
| Sorbitan stearate | | 0.8 | |
| C₁₂₋₁₅ alkyl benzoate | 5 | | |
| C₁₂₋₁₃ alkyl tartrate | | | 7 |
| Butylene glycol | | 6 | |
| dicaprylate/dicaprate | | | |
| Dicaprylyl Ether | | | 2 |
| Cyclomethicone | | 2 | 3 |
| Butylene glycol | 1 | | |
| Isohexadecane | 2 | | |
| Methylpropanediol | | | |
| Propylene glycol | | | 5 |
| Glycerine | 5 | 7 | |
| Formula XV (Uvasorb K2A) | 0.5 | 0.5 | 2 |
| Uvinul ® A Plus | 2 | 3 | |
| 3-(4-tert-Butylphenyl)-3-hydroxy-1- | 0.5 | 1.0 | 1.5 |
| (4-methoxyphenyl)propan-1-one | | | |
| 1-(4-tert-Butylphenyl)-3-hydroxy- | 1.5 | 1.0 | 0.5 |
| 3-(4-methoxyphenyl)propan-1- | | | |
| one | | | |
| Parsol SLX ® | | 3 | |
| Homosalate | | 5 | |
| Phenylbenzimidazolesulfonic acid | | 2 | 2 |
| Benzophenone-3 | 2 | | |
| Octyl salicylate | | 5 | |
| Octocrylene | 2 | | |
| Formula X (Tinsosorb S) | | 3 | |
| 2,2′-methylenebis(6-(2H- | | | 8 |
| benzotriazol-2-yl)-4-(1,1,3,3- | | | |
| tetramethylbutyl)phenol) | | | |
| Formula XII | 5 | | 4 |
| C8-C16 alkylpolyglycosides | 1 | | |
| Vitamin E acetate | 0.6 | 0.5 | 0.2 |
| Creatine/creatinine | | | 0.5 |
| BHT | | | 0.1 |
| Na₂H₂EDTA | 0.50 | | |
| Perfume, preservative | q.s. | q.s. | q.s. |
| Dyes, etc. | q.s. | q.s. | q.s. |
| Sodium hydroxide | q.s. | | q.s. |
| Potassium hydroxide | | q.s. | |
| Water | to 100.0 | to 100.0 | to 100.0 |

### Example XIII

### Cosmetic Foams

| Emulsion | D | E | F | G |
|---|---|---|---|---|
| Stearic acid | 2 | | | |
| Palmitic acid | | | 3 | 3 |
| Cetyl alcohol | 2 | 2 | | |
| Cetylstearyl alcohol | | | 2 | 2 |
| Stearyl alcohol | | | | |
| PEG-100 stearate | | 4 | | |
| PEG-40 stearate | 2 | | | |
| PEG-20 stearate | | | 3 | 3 |
| Sorbitanstearate | 0.8 | | | |
| Tridecyl trimellitate | | 5 | | |
| C₁₂₋₁₅ alkyl benzoate | | | 3 | 3 |
| Butylene glycol | 8 | | | |
| dicaprylate/dicaprate | | | | |
| Octyldodecanol | | 2 | | |
| Cocoglyceride | | | | 2 |
| Dicaprylyl Ether | | | 2 | 2 |
| Cyclomethicone | | | | |
| Dimethicone | 1 | | 2 | 2 |
| Isohexadecane | | 3 | | |
| Methylpropanediol | | 4 | | |
| Propylene glycol | | | | |
| Glycerine | 5 | | 6 | 6 |
| NeoHeliopan ® AP | | 2 | | |
| Phenylbenzimidazole- | 1 | | | 1 |
| sulfonic acid | | | | |
| 3-(4-tert-Butylphenyl)-3- | | 1.5 | 3.0 | 6.0 |
| hydroxy-1-(4-methoxy- | | | | |
| phenyl)propan-1-one | | | | |
| 1-(4-tert-Butylphenyl)-3- | 2.0 | 2.0 | 2.0 | 2.0 |
| hydroxy-3-(4-methoxy- | | | | |
| phenyl)propan-1-one | | | | |
| Ethylhexyl methoxy- | 5 | | 4 | 4 |
| cinnamate | | | | |
| Formula XVII (Uvinul T150) | | 2 | | 1 |
| Eusolex T-AVO ® | 2 | | | |
| Formula XVI (Uvasorb | 1 | | 1.0 | |
| Heb) | | | | |
| Butylmethoxydibenzoyl- | 2.5 | | 2 | 2 |
| methane | | | | |
| Formula X (Tinsoreb S) | 2 | 1.5 | 1.0 | |
| Vitamin E acetate | 0.2 | | 0.3 | 0.3 |
| Na₂H₂EDTA | | | | |
| Perfume, preservative | | | | |
| Dyes, etc. | | | | |
| Sodium hydroxide | | q-s. | q.s. | |
| Triethanolamine | q.s. | | | q.s. |
| Water | to 100.0 | to 100.0 | to 100.0 | to 100.0 |

1. A method of accelerating a photo induced reaction comprising using an accelerator in said reaction, wherein said accelerator is at least one compound of the formula I 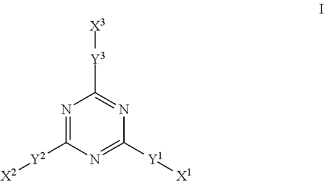
where Y¹, Y² and Y³ each stand, independently of one another, for a single bond or NH,
where X¹, X² and X³ each stand, independently of one another, for Alk¹ or a substituent of the formula II or III 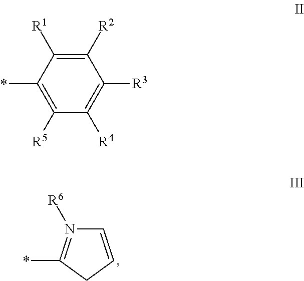
where R¹ to R⁵ each stand, independently of one another, for H, OH, Hal, Alk¹, OAlk¹, SAlk¹, NHAlk¹, N(Alk¹)₂, COOAlk¹, COOH, C(O)H, CONHAlk¹, CONH₂, COO⁻Kt⁺, Cyc¹, OCyc¹, Arl¹, OArl¹, COOArl¹, biphenylyl, Het¹, OHet¹, Si(Alk²)₃, OEth¹, COOEth¹ or for a substituent of the formula IV 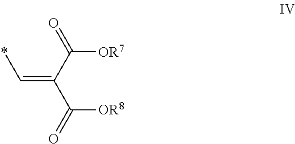
where Alk¹ in each case stands, independently of one another, for a straight-chain or branched C₁- to C₂₀-alkyl group or for a straight-chain or branched C₂- to C₂₀-alkenyl group, which may have at least one double bond, or for a straight-chain or branched C₂- to C₂₀-alkynyl group, which may have at least one triple bond, and/or in which at least one or more non-adjacent C atoms of the alkyl, alkenyl or alkynyl group may be replaced by O or trimethylsilyl and/or which may contain at least one OH bonded to a primary or secondary C atom,
where Cyc¹ in each case stands, independently of one another, for a C₃- to C₈-cycloalkyl group, which may have at least one double bond, and/or in which at least one CH₂ may be replaced by O or NH,
where Arl¹ in each case stands, independently of one another,
for an unsubstituted, mono- or polysubstituted C₆- to C₂₀-aryl group,
where Het¹ in each case stands, independently of one another, for an unsubstituted, mono- or polysubstituted C₅- to C₂₀-aryl group, in which at least one CH₂ has been replaced by O, S or NH,
where Alk² in each case stands, independently of one another,
for a straight-chain or branched C₁- to C₁₂-alkyl group, which may be interrupted by at least one O,
where R⁶ stands for Alk¹,
where R⁷ and R⁸ each stand, independently of one another,
for Alk¹ or Arl¹,
where Eth¹ in each case stands, independently of one another,
for —(CH₂—CH₂—O)ₘ₁-Alk² where 1≦m1≦16,
where Hal stands for F, Cl, Br or I,
where Kt⁺ stands for Li⁺, Na⁺ or K⁺,
and/or salt thereof as accelerator for a photoinduced reaction of 3-(4-tert-butylphenyl)-3-hydroxy-1-(4-methoxyphenyl)propan-1-one, 1-(4-tert-butyl-phenyl)-3-hydroxy-3-(4-methoxyphenyl)propan-1-one or a mixture of these two compounds to give 1-(4-tert-butylphenyl)-3-(4-methoxyphenyl)propane-1,3-dione.

2. Method according to claim 1,
characterised in that, in formula I, X¹ and X² each stand, independently of one another, for a substituent of the formula II and X³ stands for Alk¹ or, independently of X¹ and X², for a substituent of the formula II.

3. Method according to claim 1, characterised in that the substituents R² and R⁵ in formula II denote H.

4. Method according to claim 1,
characterised in that the substituents R¹, R³ and R⁴ each stand, independently of one another, for H OH, Alk¹, OAlk¹, Arl¹, Het¹, biphenylyl, COOAlk¹, CONHAlk¹ or a substituent of the formula IV

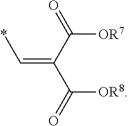

5. Method according to claim 1, characterised in that, in formula I, Y³—X³ in each case, independently of one another, comprises the combinations selected from the group Y³ stands for NH and X³ stands for Alk¹, Y³ stands for a single bond and X³ stands for a substituent of the formula II, where R¹, R², R⁴ and R⁵ denote H and R³ denotes OAlk¹, Arl¹ or biphenylyl, or Y³ stands for NH and X³ stands for a substituent of the formula II, where R¹ denotes H or Alk¹, R² and R⁵ denote H, R⁴ denotes H or OH and R³ denotes COOAlk¹, CONHAlk¹, Het¹ or a substituent of the formula IV, where R⁷ or R⁸ each, independently of one another, denote Alk¹.

6. Method according to claim 1, characterised in that, in formula I, Y¹—X¹ and Y²—X² each, independently of one another, comprises the combinations selected from the group Y¹ and Y² stand for a single bond and X¹ and X² each stand, independently of one another, for a substituent of the formula II, where R¹ denotes H, OAlk¹ or OH, R², R⁴ and R⁵ denote H and R³ denotes Arl¹, OAlk¹, Alk¹ or biphenylyl, or Y¹ and Y² stand for NH and X¹ and X² each stand, independently of one another, for a substituent of the formula II, where R¹ denotes H or OH, R² and R⁵ denote H, R⁴ denotes H or Alk¹ and R³ denotes COOAlk¹, Het¹ or a substituent of the formula IV, where R⁷ or R⁵ each, independently of one another, denote Alk¹.

7. Use of at least one compound of the formula I, as described in claim 1, together with 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or a mixture of these two compounds for the preparation of cosmetic and/or dermatological and/or pharmaceutical compositions and/or of foods and/or of food supplements and/or plastics.

8. Method according to claim 1,
characterised in that
3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or a mixture of these two compounds together with at least one compound of the formula I is used in combination with at least one further UV filter.

9. Use of a mixture of at least one compound of the formula I, as described in claim 1, together with 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or a mixture of these two compounds, as photoinduced-antioxidative system.

10. Use according to claim 9,
characterised in that the mixture is used as photoinduced stabiliser for a UV filter.

11. Use of a mixture of at least one compound of the formula I, as described in claim 1, together with 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or a mixture of these two compounds and at least one further UV filter as UV filter system for a composition, where the composition provided with a UV filter system of this type is distinguished by essentially constant or increasing UV absorption capacity on irradiation with sunlight or sun-like light in a predetermined period.

12. Composition comprising at least one vehicle which is suitable for cosmetic, pharmaceutical, dermatological compositions, foods, food supplements, household products or plastics, optionally a further UV filter and a mixture of at least one compound of the formula I, as described in claim 1, and 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or a mixture of these two compounds.

13. Composition according to claim 12,
characterised in that the composition has at most a UV absorption capacity differing by +/−5% during irradiation with sunlight or sun-like light within a predetermined period.

14. Composition according to claim 12, characterised in that the composition has increasing UV absorption capacity during irradiation with sunlight or sun-like light within a predetermined period.

15. Method for the stabilisation of the UV absorption capacity of a composition by means of a mixture of at least one compound of the formula I, as described in claim 1, together with 3-(4-tert-butylphenyl)-3-hydroxy-1-(4-methoxyphenyl)propan-1-one, 1-(4-tert-butylphenyl)-3-hydroxy-3-(4-methoxyphenyl)propan-1-one or a mixture of these two compounds, where the mixture and the composition are matched to one another in such a way that the UV absorption capacity of the composition has a maximum deviation of +/−5% or increases in a predetermined period on irradiation.
